SEQUENCE LISTING (32 sequences)

SEQ ID NO: 1 - DNA, 1929 bp, Homo sapiens
  CDS (70)...(1929)
tacggccataaagaatgctgatgggagaaccattttcctaattttcaaattgttgagctgtttgccataatggatgatcagcaagctttgaactcaatcatgcaagatttggctgtccttcataaggccagtcgaccagcattatccttgcaggaaaccagaaaagcaaaatcttcatcaccaaaaaaacagaatgatgtccgagtcaaatttgaacatagaggagaaaaaagaatccttcagttccccagaccagttaaactggaagatctgagatctaaagctaaaattgcctttggacagtctatggatctacattataccaataacgagttggtaattccattaactactcaagatgacttggacaaagctgtggaactgctggatcgtagtattcatatgaagagcctcaagatattacttgtaataaatggaagtacacaggctactaatttagaaccattgccatcactagaagatttggataatacagtatttggagcagagaggaaaaaacggctatctataataggtcctactagtagagatagaagttctcctcccccaggttacattccagatgaattacaccaggttgcccggaatgggtcattcactagtatcaacagtgaaggagagttcattccagagagcatggaccaaatgctggatccattatctttaagcagccctgaaaattctggctcaggaagttgtccatcacttgatagtcctttggatggagagagctatccaaaatcacgaatgcctagggctcagagctacccagataatcatcaggaattttcagactatgataaccctatctttgagaaatttggaaaaggaggaacatatccaagaaggtatcatgtttcatatcatcatcaagagtataatgatggtcgtaaaacttttccaagagctagaaggacccaggggaccagcttacggtctcctgtgagtttcagtcctactgatcattccttaagcactagtagtggaagcagtatctttaccccagagtatgatgatagtcgaataagaagaaggggaagtgacatagacaatcctactttgaccgtaatggacatcagcccacccagccgttcacctcgagctccgaccaactggagattgggcaaactgcttggccaaggagcctttggaagggtctacctctgttatgatgttgatacaggaagagaattggctgttaagcaagttcaatttgaccccgatagtcctgagaccagcaaggaagtaaatgcacttgagtgtgaaattcagttgctgaaaaacttgctacatgagcgaattgttcagtattatggctgtttgagggatccccaggaaaaaacactttccatatttatggaatatatgccagggggttcaattaaggaccaattaaaagcatatggcgctcttactgagaatgtgactaggaaatacacccgtcagattctggagggtgtccattatttgcacagtaatatgattgtccatagagatatcaaaggcgcaaatatcctgcgagattcaacaggcaacgtcaaactaggagattttggggccagcaaacggcttcagaccatctgtctctcagggacaggaatgaagtctgtcacgggcacaccatactggatgagccctgaagtcatcagtggagaaggctatggaagaaaagcagacatctggagtgttgcatgtactgtggtagaaatgctaactgaaaagccgccttgggctgaatttgaagcaatggctgccatctttaaaatcgccactcagccaacaaacccaaagctgccacctcatgtctcagactatactcgagatttcctcaaacggatttttgtagaggccaaactgagaccttcagctgatgaactcttaaggcacatgtttgtgcattatcactag

SEQ ID NO: 2 - protein, 619 aa, Homo sapiens
MDDQQALNSIMQDLAVLHKASRPALSLQETRKAKSSSPKKQNDVRVKFEHRGEKRILQFPRPVKLEDLRSKAKIAFGQSMDLHYTNNELVIPLTTQDDLDKAVELLDRSIHMKSLKILLVINGSTQATNLEPLPSLEDLDNTVFGAERKKRLSIIGPTSRDRSSPPPGYIPDELHQVARNGSFTSINSEGEFIPESMDQMLDPLSLSSPENSGSGSCPSLDSPLDGESYPKSRMPRAQSYPDNHQEFSDYDNPIFEKFGKGGTYPRRYHVSYHHQEYNDGRKTFPRARRTQGTSLRSPVSFSPTDHSLSTSSGSSIFTPEYDDSRIRRRGSDIDNPTLTVMDISPPSRSPRAPTNWRLGKLLGQGAFGRVYLCYDVDTGRELAVKQVQFDPDSPETSKEVNALECEIQLLKNLLHERIVQYYGCLRDPQEKTLSIFMEYMPGGSIKDQLKAYGALTENVTRKYTRQILEGVHYLHSNMIVHRDIKGANILRDSTGNVKLGDFGASKRLQTICLSGTGMKSVTGTPYWMSPEVISGEGYGRKADIWSVACTVVEMLTEKPPWAEFEAMAAIFKIATQPTNPKLPPHVSDYTRDFLKRIFVEAKLRPSADELLRHMFVHYH

SEQ ID NO: 3 - DNA, 1857 bp, Homo sapiens
  CDS (1)...(1857)
atggatgatcagcaagctttgaactcaatcatgcaagatttggctgtccttcataaggccagtcgaccagcattatccttgcaggaaaccagaaaagcaaaatcttcatcaccaaaaaaacagaatgatgtccgagtcaaatttgaacatagaggagaaaaaagaatccttcagttccccagaccagttaaactggaagatctgagatctaaagctaaaattgcctttggacagtctatggatctacattataccaataacgagttggtaattccattaactactcaagatgacttggacaaagctgtggaactgctggatcgtagtattcatatgaagagcctcaagatattacttgtaataaatggaagtacacaggctactaatttagaaccattgccatcactagaagatttggataatacagtatttggagcagagaggaaaaaacggctatctataataggtcctactagtagagatagaagttctcctcccccaggttacattccagatgaattacaccaggttgcccggaatgggtcattcactagtatcaacagtgaaggagagttcattccagagagcatggaccaaatgctggatccattatctttaagcagccctgaaaattctggctcaggaagttgtccatcacttgatagtcctttggatggagagagctatccaaaatcacgaatgcctagggctcagagctacccagataatcatcaggaattttcagactatgataaccctatctttgagaaatttggaaaaggaggaacatatccaagaaggtatcatgtttcatatcatcatcaagagtataatgatggtcgtaaaacttttccaagagctagaaggacccaggggaccagcttacggtctcctgtgagtttcagtcctactgatcattccttaagcactagtagtggaagcagtatctttaccccagagtatgatgatagtcgaataagaagaaggggaagtgacatagacaatcctactttgaccgtaatggacatcagcccacccagccgttcacctcgagctccgaccaactggagattgggcaaactgcttggccaaggagcctttggaagggtctacctctgttatgatgttgatacaggaagagaattggctgttaagcaagttcaatttgaccccgatagtcctgagaccagcaaggaagtaaatgcacttgagtgtgaaattcagttgctgaaaaacttgctacatgagcgaattgttcagtattatggctgtttgagggatccccaggaaaaaacactttccatatttatggaatatatgccagggggttcaattaaggaccaattaaaagcatatggcgctcttactgagaatgtgactaggaaatacacccgtcagattctggagggtgtccattatttgcacagtaatatgattgtccatagagatatcaaaggcgcaaatatcctgcgagattcaacaggcaacgtcaaactaggagattttggggccagcaaacggcttcagaccatctgtctctcagggacaggaatgaagtctgtcacgggcacaccatactggatgagccctgaagtcatcagtggagaaggctatggaagaaaagcagacatctggagtgttgcatgtactgtggtagaaatgctaactgaaaagccgccttgggctgaatttgaagcaatggctgccatctttaaaatcgccactcagccaacaaacccaaagctgccacctcatgtctcagactatactcgagatttcctcaaacggatttttgtagaggccaaactgagaccttcagctgatgaactcttaaggcacatgtttgtgcattatcac

SEQ ID NO: 4 - DNA, 2133 bp, Homo sapiens
  CDS (102)...(1958)
  misc_feature 86: n = A,T,C or G
ttttattaatatgctgatctgttttatcttctcacggccataaagaatgctgattggagaaccattttcctaattttcaaattgttnagctgtttgccataatggatgatcagcaagctttgaactcaatcatgcaagatttggctgtccttcataaggccagtcgaccagcattatccttgcaggaaaccagaaaagcaaaatcttcatcaccaaaaaaacagaatgatgtccgagtcaaatttgaacatagaggagaaaaaagaatccttcagttccccagaccagttaaactggaagatctgagatctaaagctaaaattgcctttggacagtctatggatctacattataccaataacgagttggtaattccattaactactcaagatgacttggacaaagctctggaactgctggatcgtagtattcatatgaagagcctcaagatattacttgtaataaatggaagtacacaggctactaatttagaaccattgccatcactagaagatttggataatacagtatttggagcagagaggaaaaaacggctatctataataggtcctactagtagagatagaagttctcctcccccgggttacattccagatgaattacaccaggttgcccggaatgggtcattcactagtatcaacagtgaaggagagttcattccagagagcatggaacaaatgctggatccattatctttaagcagccctgaaaattctggctcaggaagttgtccatcacttgatagtcctttgggtggagagagctatccaaaatcacgaatgcctagggctcagagctacccagataatcatcaggaattttcagactatgataaccctatctttgagaaatttggaaaaggaggaacatatccaagaaggtatcatgtttcatatcatcataaggataatgatggtcgtaaaacttttccaagagctagaaggacccaggggaaccagcttacgtctcctgtgagtttcagtcctactgatcattccttaagcactagtagtggaagcagtatctttaccccagagtatgatgatagtcgaataagaagaaggggaagtgacatagacaatcctactttgacagtaatggacatcagcccacccagccgttcacctcgagctccgaccaactggagattgggcaaactgcttggccaaggagcctttggaagggtctacctctgttatgatgttgatacaggaagagaattggctgttaagcaagttcaatttgaccccgatagtcctgagaccagcaaggaagtaaatgcacttgagtgtgaaattcagttgctgaaaaactttctacatgagcgaattgttcagtattatggctgtttgagggatccccaggaaaaaacactttccatatttatggaatatatgccagggggttcaattaaggaccaattaaaagcatatggcgctcttactgagaatggtactaggaaatacacccgtcagattctggagggtgtccattatttgcacagtaatatgattctccaccgcgacatcaaaggcgcaaatatccttcgagattcaacaggcaacgtcaaactaggagattttggggccagcaaacggcttcagaccatctgtctctcagggacaggaatgaagtctgtcacgggcacaccatactggatgagccctgaagtcatcagtggacaaggctatggaagaaaagcagacatctggagtgttgcatgtaccgtggtagaaatgctaactgaaaagccgccttgggctgaatttgaagcaatggctgccatctttaaaatcgccactcagccaacaaacccaaagctgccacctcatgtctcagactatactcgagatttcctcaaacggatttttgtagaggccaaactgagaccttcagctgatgaactcttaaggcacatgtttgtgcattatcactagcagccagtaacctctcctgtgcctctacctagctcccatctattcattcaccttctctctgactgcacttttcttttttataaaaaaagagagagggggagaaaaaagacaagagggaaagtatttctcttgattcttggttaaattgttaataataataatatcctaatttata

SEQ ID NO: 5 - protein, 618 aa, Homo sapiens [sequence omitted]

SEQ ID NO: 6 - protein, 619 aa, Homo sapiens
MDDQQALNSIMQDLAVLHKASRPALSLQETRKAKSSSPKKQNDVRVKFEHRGEKRILQFPRPVKLEDLRSKAKIAFGQSMDLHYTNNELVIPLTTQDDLDKALELLDRSIHMKSLKILLVINGSTQATNLEPLPSLEDLNNTVFGAERKKRLSVIGPTNRDRSSPPPGYIPDELHQVARNGSFTSINSEGEFIPESMNQMLDPLSLSSPENSGSGSCPSLDSPLGGESYPKSRMPRAQSYPDNHQEFSDYDNPIFEKFGKGGTYPRRYHVSYHHKYDNDGRKTFPRARRTQGNQLTSPVSFSPTDHSLSTSSGSSIFTPEYDDSRIRRRGSDIDNPTLTVMDISPPSRSPRAPTNWRLGKLLGQGAFGRVYLCYDVDTGRELAVKQVQFDPDSPETSKEVNALECEIQLLKNLLHERIVQYYGCLRDPQEKTLSIFMEYMPGGSIKDQLKAYGALTENVTRKYTRQILEGVHYLHSNMIVHRDIKGANILRDSTGNVKLGDFGASKRLQTICLSGTGMKSVTGTPYWMSPEVISGEGYGRKADIWSVACTVVEMLTEKPPWAEFEAMAAIFKIATQPTNPKLPPHVSDYTRDFLKRIFVEAKLRPSAEELLRHMFVHYH

SEQ ID NO: 7 - DNA, 2013 bp, Homo sapiens
  CDS (124)...(1983)
ggccagctcggtggcctcctctcggccctcggctccgcgatccccgcccagcggccgggcaataaagaatgttgatgggagaaccattttcctaattttcaaattattgagctggtcgccataatggatgatcagcaagctttgaattcaatcatgcaagatttggctgtccttcataaggccagtcggccagcattatctttacaagaaaccaggaaagcaaaaccttcatcaccaaaaaaacagaatgatgttcgagtcaaatttgaacatagaggagaaaaaaggatcctgcaggttactagaccagttaaactagaagacctgagatctaagtctaagatcgcctttgggcagtctatggatctacactataccaacaatgagttggtaattccgttaactacccaagatgacttggacaaagctgtggaactgctggatcgcagtattcacatgaagagtctcaagatattacttgtagtaaatgggagtacacaggctactaatttagaaccatcaccgtcaccagaagatttgaataatacaccacttggtgcagagaggaaaaagcggctatctgtagtaggtccccctaatagggatagaagttcccctcctccaggatacattccagacgagctacaccagattgcccggaatgggtcattcactagcatcaacagtgaaggagagttcattccagagagcatggaccaaatgctggatccattgtctttaagcagccctgaaaattctggctcaggaagctgtccgtcacttgatagtcctttggatggagaaagctacccaaaatcacggatgcctagggcacagagctacccagataatcatcaggagtttacagactatgataaccccatttttgagaaatttggaaaaggaggaacatatccaagaaggtaccacgtttcctatcatcaccaggagtataatgacggtcggaagacttttccaagagctagaaggacccagggcaccagtttccggtctcctgtgagcttcagtcctactgatcactccttaagcaatagtagtggaagcagtgtctttaccccagagtatgacgacagtcgaatgagaagacgggggagtgacatagacaaccctactttgactgtcacagacatcagcccaccatgccgttcacctcgagctccgaccaactggagactgggcaagctgcttggccaaggagattttggtagggtctacctctgctatgatgttgataccggaagagagctggctgttaagcaagttcagtttaaccctgagagcccagagaccagcaaggaagtaaatgcacttgagtgtgaaattcagttgttgaaaaacttgttgcatgagcgaattgttcagtattatggctgtttgagggatcctcaggagaaaacactttccatctttatggagtatatgccagggggttcaattaaggaccaactaaaagcctacggagctcttactgagaacgtgacgaggaagtacacccgtcagattctggagggggtccattatttgcatagtaatatgattgtccatagagatatcaaaggagcaaatatcttaagggattccacaggcaatatcaagttaggagactttggggctagtaaacggcttcagaccatctgtctctcaggcacaggaatgaagtctgtcacaggcacgccatactggatgagtcctgaggtcatcagtggagaaggctatggaagaaaagcagacatctggagtgtagcatgtagagtggtagaaatgctaactgaaaagccaccttgggctgaatttgaagcaatggctgccatctttaagatcgccactcagccaacgaacccaaagctgccacctcatgtctcagactatactcgggacttcctcaaacggatttttgtagaggccaaacttcgaccttcagcggaggagctcttgcggcacatgtttgtgcattatcactagcatcggcggcttcggtcctccaccatctcc

SEQ ID NO: 8 - protein, 619 aa, Homo sapiens
MDDQQALNSIMQDLAVLHKASRPALSLQETRKAKPSSPKKQNDVRVKFEHRGEKRILQVTRPVKLEDLRSKSKIAFGQSMDLHYTNNELVIPLTTQDDLDKAVELLDRSIHMKSLKILLVVNGSTQATNLEPSPSPEDLNNTPLGAERKKRLSVVGPPNRDRSSPPPGYIPDELHQIARNGSFTSINSEGEFIPESMDQMLDPLSLSSPENSGSGSCPSLDSPLDGESYPKSRMPRAQSYPDNHQEFTDYDNPIFEKFGKGGTYPRRYHVSYHHQEYNDGRKTFPRARRTQGTSFRSPVSFSPTDHSLSNSSGSSVFTPEYDDSRMRRRGSDIDNPTLTVTDISPPCRSPRAPTNWRLGKLLGQGDFGRVYLCYDVDTGRELAVKQVQFNPESPETSKEVNALECEIQLLKNLLHERIVQYYGCLRDPQEKTLSIFMEYMPGGSIKDQLKAYGALTENVTRKYTRQILEGVHYLHSNMIVHRDIKGANILRDSTGNIKLGDFGASKRLQTICLSGTGMKSVTGTPYWMSPEVISGEGYGRKADIWSVACRVVEMLTEKPPWAEFEAMAAIFKIATQPTNPKLPPHVSDYTRDFLKRIFVEAKLRPSAEELLRHMFVHYH

SEQ ID NO: 9 - DNA, 2003 bp, Homo sapiens
  misc_feature 17: n = A,T,C or G
taattttcaaattgttnagctgtttgccataatggatgatcagcaagctttgaactcaatcatgcaagatttggctgtccttcataaggccagtcgaccagcattatccttgcaggaaaccagaaaagcaaaatcttcatcaccaaaaaaacagaatgatgtccgagtcaaatttgaacatagaggagaaaaaagaatccttcagttccccagaccagttaaactggaagatctgagatctaaagctaaaattgcctttggacagtctatggatctacattataccaataacgagttggtaattccattaactactcaagatgacttggacaaagctctggaactgctggatcgtagtattcatatgaagagcctcaagatattacttgtaataaatggaagtacacaggctactaatttagaaccattgccatcactagaagatttggataatacagtatttggagcagagaggaaaaaacggctatctataataggtcctactagtagagatagaagttctcctcccccgggttacattccagatgaattacaccaggttgcccggaatgggtcattcactagtatcaacagtgaaggagagttcattccagagagcatggaacaaatgctggatccattatctttaagcagccctgaaaattctggctcaggaagttgtccatcacttgatagtcctttggatggagagagctatccaaaatcacgaatgcctagggctcagagctacccagataatcatcaggaattttcagactatgataaccctatctttgagaaatttggaaaaggaggaacatatccaagaaggtatcatgtttcatatcatcatcaagagtataatgatggtcgtaaaacttttccaagagctagaaggacccaggggaaccagcttacgtctcctgtgagtttcagtcctactgatcattccttaagcactagtagtggaagcagtatctttaccccagagtatgatgatagtcgaataagaagaaggggaagtgacatagacaatcctactttgacagtaatggacatcagcccacccagccgttcacctcgagctccgaccaactggagattgggcaaactgcttggccaaggagcctttggaagggtctacctctgttatgatgttgatacaggaagagaattggctgttaagcaagttcaatttgaccccgatagtcctgagaccagcaaggaagtaaatgcacttgagtgtgaaattcagttgctgaaaaacttgctacatgagcgaattgttcagtattatggctgtttgagggatccccaggaaaaaacactttccatatttatggaatatatgccagggggttcaattaaggaccaattaaaagcatatggcgctcttactgagaatgtgactaggaaatacacccgtcagattctggagggtgtccattatttgcacagtaatatgattgtccatagagatatcaaaggcgcaaatatccttcgagattcaacaggcaacgtcaaactaggagattttggggccagcaaacggcttcagaccatctgtctctcagggacaggaatgaagtctgtcacgggcacaccatactggatgagccctgaagtcatcagtggacaaggctatggaagaaaagcagacatctggagtgttgcatgtaccgtggtagaaatgctaactgaaaagccgccttgggctgaatttgaagcaatggctgccatctttaaaatcgccactcagccaacaaacccaaagctgccacctcatgtctcagactatactcgagatttcctcaaacggatttttgtagaggccaaactgagaccttcagctgatgaactcttaaggcacatgtttgtgcattatcactagcagccagtaacctctcctgtgcctctacctagctcccatctattcattcaccttctctctgactgcacttttcttttttataaaaaaagagagagggggagaaaaaagacaa

SEQ ID NO: 10 - protein, 619 aa, Homo sapiens [sequence omitted]

SEQ ID NO: 11 - protein, 34 aa, Artificial Sequence
  consensus sequence
XGXGXXXXXXXXXXXXXXXXXXXXXXXXXXXXXK

SEQ ID NO: 12 - protein, 13 aa, Artificial Sequence
  consensus sequence
XXXXDXKXXNXXX

SEQ ID NO: 13 - DNA, 22 bp, Artificial Sequence
  Primer
acggccataaagaatgctgatt

SEQ ID NO: 14 - DNA, 20 bp, Artificial Sequence
  Primer
tgcccaatctccagttggtc

SEQ ID NO: 15 - DNA, 24 bp, Artificial Sequence
  Primer
agaaggggaagtgacatagacaat

SEQ ID NO: 16 - DNA, 24 bp, Artificial Sequence
  Primer
taacaatttaaccaagaatcaaga

SEQ ID NO: 17 - DNA, 18 bp, Artificial Sequence
  Primer
aaacaagttcaatttgac

SEQ ID NO: 18 - DNA, 18 bp, Artificial Sequence
  Primer
atcggggtcaaattgaac

SEQ ID NO: 19 - DNA, 18 bp, Artificial Sequence
  Primer
accaataacgcgttggta

SEQ ID NO: 20 - DNA, 18 bp, Artificial Sequence
  Primer
tcacaggagacgtaagct

SEQ ID NO: 21 - DNA, 19 bp, Artificial Sequence
  Primer
agccaaatcttgcatgatt

SEQ ID NO: 22 - DNA, 19 bp, Artificial Sequence
  Primer
actatactcgagatttcct

SEQ ID NO: 23 - DNA, 18 bp, Artificial Sequence
  Primer
atggatgatcagcaagct

SEQ ID NO: 24 - DNA, 18 bp, Artificial Sequence
  Primer
attataccaataacgagt

SEQ ID NO: 25 - DNA, 18 bp, Artificial Sequence
  Primer
tctataataggtcctact

SEQ ID NO: 26 - DNA, 18 bp, Artificial Sequence
  Primer
caaaatcacgaatgccta

SEQ ID NO: 27 - DNA, 18 bp, Artificial Sequence
  Primer
acaatcctactttgaacc

SEQ ID NO: 28 - DNA, 18 bp, Artificial Sequence
  Primer
actttccatatttatgga

SEQ ID NO: 29 - DNA, 18 bp, Artificial Sequence
  Primer
tggcagctttgggtttgt

SEQ ID NO: 30 - DNA, 17 bp, Artificial Sequence
  Primer
tctgacgggtgtatttc

SEQ ID NO: 31 - DNA, 18 bp, Artificial Sequence
  Primer
actcacaggagaccgtaa

SEQ ID NO: 32 - DNA, 18 bp, Artificial Sequence
  Primer
atggcaatggttctaaat

(19) United States
(12) Patent Application Publication
(10) Pub. No.: US 2004/0019918 A1
(43) Pub. Date: Jan. 29, 2004

(54) Human MEKK2 protein and nucleic acid molecules and uses therefor

(75) Inventors: Anne M. Whalen, Suwanee, GA (US); Christopher K. Cook, Alpharetta, GA (US); James A. Sikorski, Atlanta, GA (US)

Correspondence Address: LAHIVE & COCKFIELD, 28 STATE STREET, BOSTON, MA, 02109, US

(73) Assignee: AtheroGenics, Inc., Alpharetta, GA (US)

(21) Appl. No.: 10/456,422

(22) Filed: Jun. 5, 2003

Related U.S. Application Data
(60) Provisional application No. 60/453,712, filed on Jun. 5, 2002.

(51) Int. Cl. A01K 67/00; C12Q 1/68; C12P 21/06; C12N 9/12; C07H 21/04; C12N 5/06

(52) U.S. Cl. 800/8; 435/6; 435/69.1; 435/320.1; 435/325; 435/194; 536/23.2

(57) ABSTRACT

Isolated nucleic acid molecules encoding human MEKK2, and isolated human MEKK2 proteins, are provided. The invention further provides antisense nucleic acid molecules, recombinant expression vectors containing a nucleic acid molecule of the invention, host cells into which the expression vectors have been introduced and non-human transgenic animals carrying a human MEKK2 transgene. The invention further provides human MEKK2 fusion proteins and anti-human MEKK2 antibodies. Methods of using the human MEKK2 proteins and nucleic acid molecules of the invention are also disclosed, including methods for detecting human MEKK2 activity in a biological sample, methods of modulating human MEKK2 activity in a cell, and methods for identifying agents that modulate the activity of human MEKK2.

### RELATED APPLICATIONS

This application claims priority to U.S. provisional Application No. ______, formerly U.S. Ser. No. 10/163,811, filed on Jun. 5, 2002, the content of which is incorporated herein in its entirety by this reference.

### FIELD OF THE INVENTION

This invention relates to isolated MEKK2 nucleic acid and protein molecules and uses for said molecules.

### BACKGROUND OF THE INVENTION

MEKK2 (also known as mitogen-activated protein kinase kinase kinase 2, MAP3K2, MEK kinase 2 and MAP/ERK kinase kinase 2) is a dual specific serine/threonine kinase that functions to mediate cellular responses to mitogenic stimuli. MEKK2 was originally isolated and cloned from mouse NIH3T3 cells (Blank et al. (1996) J. Biol. Chem. 271:5361-5368). Murine MEKK2 is further described in U.S. Pat. No. 5,854,043 which teaches the regulatory and catalytic domains of the protein. Expression of murine MEKK2 transfected into HEK293 cells was shown to activate the JNK/SAPK and p42/44 MAPK pathways. Within these pathways, MEKK2 was shown to phosphorylate JNK kinase and MEK1, each a downstream kinase of the JNK/SAPK and p42/44 MAPK pathways, respectively. (Blank et al., supra and Deacon and Blank (1997) J. Biol. Chem. 272:14489-14496). MEKK2 has also been shown to interact with 14-3-3 proteins via the kinase domain in the absence of any phosphorylation events. This protein-protein interaction may represent a non-enzymatic mechanism for controlling the effects of MEKK2 in the MAP kinase pathway (Fanger et al. (1998) J. Biol. Chem. 273:3476-3483).

MEKK2 has been implicated in inflammatory responses. In particular, Zhao and Lee have shown that MEKK2 can activate the NF-κB pathway in HeLa cells by phosphorylating the cytoplasmic inhibitory molecule, IκB (Zhao and Lee (1999) J Biol Chem. 274:8355-8358). Activated NF-κB is known to translocate to the nucleus affecting the transcription of several genes upon cellular induction by proinflammatory agents.

Recently the sequences of at least three human MEKK2 isoforms have been described. Johnson (U.S. Pat. No. 6,312,934) describes the cloning of a human MEKK2 cDNA from T47D breast carcinoma cells that encodes a 619 amino acid-protein having 98% identity to murine MEKK2. Su et al. describe a human MEKK2 cDNA from Jurkat T-cells that encodes a polypeptide of 618 or 619 amino acids which is highly homologous to both human and murine MEKK3 across the catalytic domains and significantly less homologous across the N-terminal noncatalytic region (Su et al. (2001) J. Biol. Chem. 276:14784-14790 and GenBank Accession No. AF111105). Northern blot analysis showed that MEKK2 was ubiquitously expressed, with the highest level in peripheral blood leukocytes. This human MEKK2 was implicated in T-cell receptor/CD3-mediated c-Jun N-terminal kinase (JNK)/mitogen-activated protein kinase (MAPK) activation and interleukin-2 gene expression. (Su et al., supra). The cDNA sequence encoding a third isoform (isolated from T cells) has been deposited in the GenBank public sequence database by Wang and Lo (Viogenics, Sunnyvale, Calif.) having GenBank Accession No. AF239798. Notably, there are discrepancies between these published MEKK2 sequences with differences of 13 to 28 residues being apparent when sequences are aligned for comparison purposes. The description in the art of multiple MEKK2 sequences serves only to confuse and undermine any interpretation of the MEKK2 crystal structure, complicates homology studies of MEKK2 with closely related kinases of known structure, and negates the use of kinase inhibitors in in silico small molecule inhibitor docking studies. Knowledge of the precise human MEKK2 sequence would facilitate structural and kinetic analyses of this enzyme, as well as the successful search for, and identification of, SMIs (small molecule inhibitors) having selectivity and specificity for this kinase.

### SUMMARY OF THE INVENTION

The present invention is based, at least in part, on the discovery of a novel human MEKK2 nucleotide sequence and corresponding amino acid sequence. The cDNA sequence was identified in Jurkat T cells and has been verified in at least two distinct normal human tissues, i.e., normal human thymus and normal human spleen, and found to be 100% identical. Accordingly, the invention provides novel human MEKK2 compositions. In particular, the invention provides isolated nucleic acid molecules encoding novel human MEKK2 proteins and/or biologically active fragments thereof. The invention further provides isolated human MEKK2 proteins and/or biologically active fragments thereof. Since the MEKK2 compositions of the invention are human-derived, they function optimally in human cells (compared with non-human MEKK2 compositions) and do not stimulate an immune response in humans.

One aspect of the invention pertains to isolated nucleic acid molecules that comprise nucleotide sequences encoding human MEKK2 proteins and/or biologically active fragments thereof. In one embodiment, the nucleic acid molecule comprises the nucleotide sequence of SEQ ID NO: 3. In another embodiment, the nucleic acid molecule has at least 99.6% identity, more preferably at least 99.7% identity, even more preferably at least 99.8% identity, and even more preferably at least 99.9% or more identity with the nucleotide sequence SEQ ID NO:3. In another embodiment, the nucleic acid molecule comprises nucleotides 1137-1917 of SEQ ID NO:3. In yet another embodiment, the nucleic acid molecule has at least 99.6% identity, more preferably at least 99.7% identity, even more preferably at least 99.8% identity, and even more preferably at least 99.9% or more identity with nucleotides 1137-1917 of SEQ ID NO:3.

The isolated nucleic acid molecules of the invention encoding human MEKK2 proteins, or bioactive fragments thereof, can be incorporated into a vector, such as an expression vector, and this vector can be introduced into a host cell. The invention also provides methods for producing human MEKK2 proteins, or bioactive fragments thereof, that involve culturing a host cell of the invention (carrying a human MEKK2 expression vector) in a suitable medium until the human MEKK2 protein or fragment is produced. The methods can further involve isolating the human MEKK2 proteins or fragments from the medium or the host cell.

Another aspect of the invention pertains to isolated human MEKK2 proteins and/or biologically active fragments thereof. In one embodiment, a human MEKK2 protein comprises the amino acid sequence of SEQ ID NO: 2. In another embodiment, the protein has at least 99.1% amino acid identity, more preferably at least 99.2% amino identity, even more preferably at least 99.3% amino acid identity, and even more preferably at least 99.4%, 99.5%, 99.6%, 99.7%, 99.8%, 99.9% or more amino acid identity with the amino acid sequence of SEQ ID NO:2. In another embodiment, a human MEKK2 fragment comprises amino acids 356-616 of SEQ ID NO:2. In yet another embodiment, a human MEKK2 fragment has at least 99.1% amino acid identity, more preferably at least 99.2% amino identity, even more preferably at least 99.3% amino acid identity, and even more preferably at least 99.4%, 99.5%, 99.6%, 99.7%, 99.8%, 99.9% or more amino acid identity with amino acid residues 356-616 of SEQ ID NO:2.

Fusion proteins, comprising a human MEKK2 protein, preferably the MEKK2 protein set forth as SEQ ID NO:2, or a bioactive fragment or functional variant thereof, operatively linked to a polypeptide other than human MEKK2, are also encompassed by the invention, as well as antibodies that specifically bind to the human MEKK2 protein, preferably the MEKK2 protein set forth as SEQ ID NO:2, or a bioactive fragment or functional variant thereof. The antibodies can be, for example, polyclonal antibodies or monoclonal antibodies. In one embodiment, the antibodies are coupled to a detectable substance.

Another aspect of the invention pertains to a nonhuman transgenic animal that contains cells carrying a transgene encoding a human MEKK2 protein, preferably the MEKK2 protein set forth as SEQ ID NO:2 or a functional variant thereof.

Yet another aspect of the invention pertains to a method for specifically detecting the presence of human MEKK2, preferably the MEKK2 protein set forth as SEQ ID NO:2 or a functional variant thereof, in a biological sample. Yet another aspect of the invention pertains to a method for specifically detecting the presence of a polynucleotide encoding a human MEKK2, preferably encoding the MEKK2 protein set forth as SEQ ID NO:2 or a functional variant thereof, in a biological sample. Still another aspect of the invention pertains to methods for identifying compounds capable of modulating the expression, activity or biological function of a human MEKK2 protein, preferably the MEKK2 protein set forth as SEQ ID NO:2 or a functional variant thereof.

### BRIEF DESCRIPTION OF THE DRAWINGS

FIGS. 1A-C depicts the nucleotide sequence of human MEKK2 (SEQ ID NO:1). Nucleotides 1-69 correspond to the 5′ untranslated region (5′ UTR) and nucleotides 70-1870 correspond to the open reading frame (ORF).

FIG. 2 depicts the amino acid sequence of human MEKK2 (SEQ ID NO:2).

FIGS. 3A-C depicts an alignment of the amino acid sequence of the human MEKK2 protein of the instant invention (MEKK2.4, SEQ ID NO:2) with four previously described human MEKK2 isoforms. Dashed boxes indicate identical amino acid residues and bold indicates homologous amino acid residues. The kinase domain is indicated by arrows. Box I indicates the “protein kinase ATP-binding region signature”. The ATP-binding lysine is indicated by an asterisk. Box II indicates the “serine/threonine protein kinase active-site signature”. The active site aspartate residue is indicated by an asterisk.

### DETAILED DESCRIPTION OF THE INVENTION

This invention pertains to human MEKK2 compositions, such as isolated nucleic acid molecules encoding human MEKK2 and isolated human MEKK2 proteins, as well as methods of use thereof. The human MEKK2 nucleic acid and protein molecules of the invention have sequences distinct from the MEKK2 sequences previously published. The nucleotide sequence was identified in Jurkat T cells and has been verified (i.e., is 100% identical to that in normal human tissues thymus and normal human spleen). It is believed that the sequences disclosed herein represent the precise human MEKK2 sequences. Recombinant proteins having the amino acid sequence set forth as SEQ ID NO:2 are believed to be a better choice for use in processes requiring recombinant human MEKK2 protein. Likewise, reagents based on the sequence information set forth as SEQ ID NO:1 are believed to be superior for use in processes requiring human MEKK2 nucleic acid reagents.

Knowledge of the precise human MEKK2 sequence also allows the skilled artisan to:

a) accurately perform in vitro and cell-based experiments to determine precise biological functions (e.g., signaling mechanism, etc.) of human MEKK2;

b) accurately define homology models of human MEKK2, for example, for comparison with other kinases;

c) differentiate the key structural features of MEKK2, for example, as compared to MEKK1, MEKK3 and MEKK4;

d) accurately perform mutagenesis studies, and create self-spicing RNA molecules and small interfering RNAs siRNAs to inhibit cellular expression in target tissues for studies on function of MEKK2;

e) unambiguously align amino acid residues with future crystal 3D structure;

f) unambiguously predict proteolytic cleavage fragments for protein analysis of active-site pocket fluid dynamics (e.g. in deuterium exchange experiments utilizing mass spectroscopy);

g) accurately predict amino acid residue interactions with small molecule inhibitors in relationships to known protein 3D structures utilizing in silico “docking” algorithms;

h) accurately modify and mutate individual residues to study enzymology and 3D structure in relationship to native enzyme, small-molecule inhibitors and interacting proteins;

i) accurately modify and mutate individual residues to optimize the expression, purification, stability and activity of the kinase and/or protein-interacting domains of the native human protein or a functional variant, in heterologous protein expression systems, including baculovirus, bacterial expression and cell-free T7-RNA polymerase protein expression systems;

This list is representative of experimental designs critical for understanding MEKK2 activity and functions in vivo and in vitro, that begin with and depend on accurate and unambiguous sequence information for development of therapeutic agents that act on MEKK2.

So that the invention may be more readily understood, certain terms are first defined.

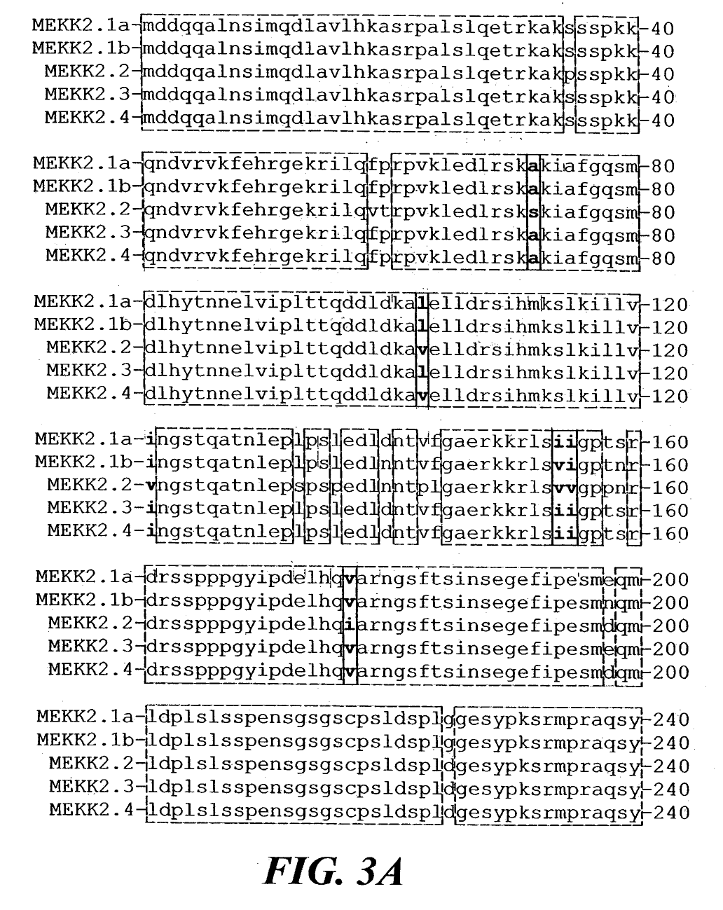

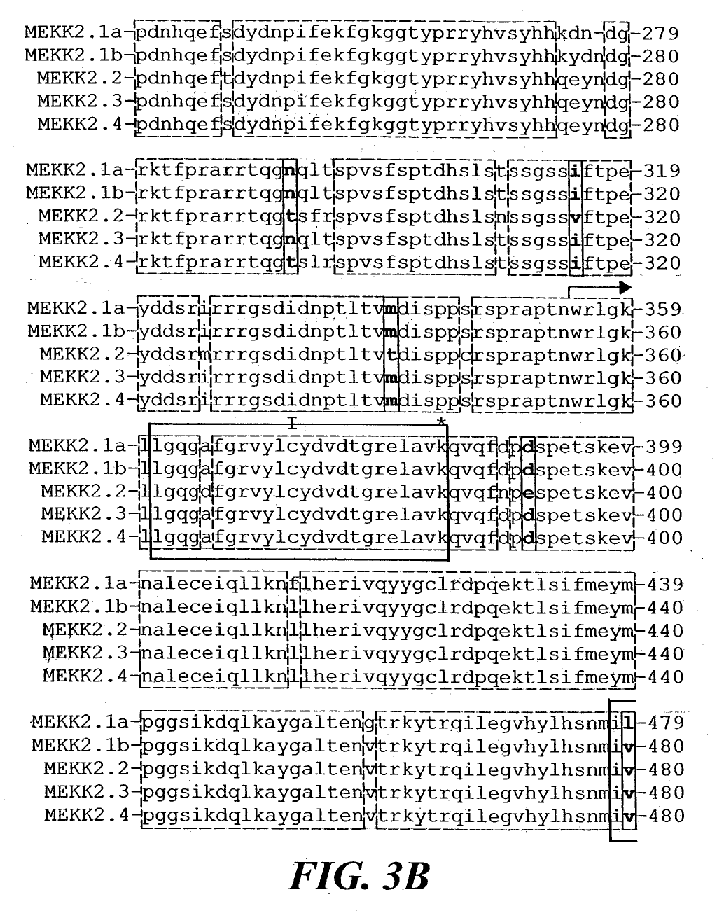

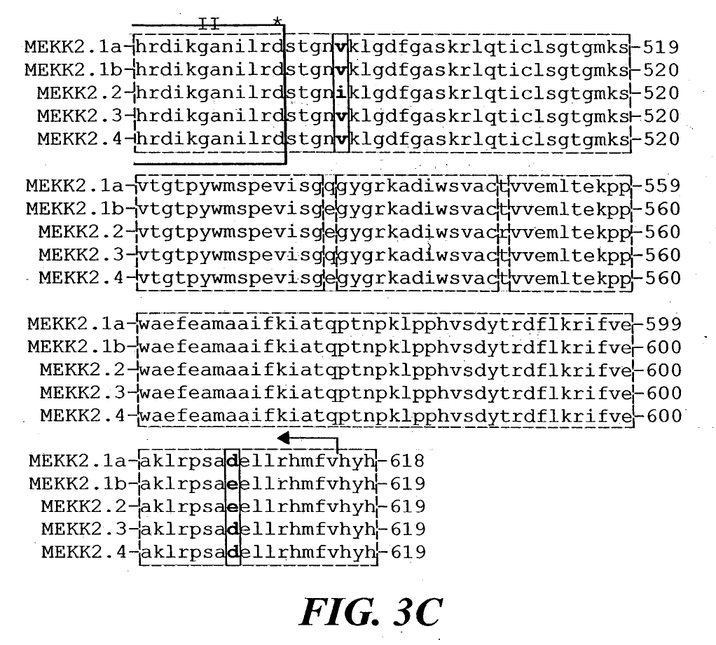

As used herein, the term “human MEKK2” is intended to encompass proteins that share the distinguishing structural and functional features (described further herein) of the human MEKK2 protein set forth as SEQ ID NO: 2, including the amino acid residues unique to the human MEKK2 set forth as SEQ ID NO:2, which are indicated clearly by the alignment depicted in FIGS. 3A-C.

The invention also includes functionally equivalent variants of the MEKK2 protein set forth as SEQ ID NO:2, as well as polynucleotides that encode such variants. A “functionally equivalent variant” is a polypeptide that differs in amino acid sequence from the MEKK2 protein set forth as SEQ ID NO:2 (i.e., the reference polypeptide) by no greater than 6 insertions, deletions, substitutions (e.g., conservative or non-conservative substitutions), or combination thereof, and retains the biological activity of the MEKK2 protein set forth as SEQ ID NO:2, as defined herein. Particularly preferred variants are those that differ from the MEKK2 protein set forth as SEQ ID NO:2 by no greater than 4-5 insertions, deletions, substitutions, or combination thereof, and retain the biological activity of the MEKK2 protein set forth as SEQ ID NO:2. Particularly preferred variants are those that differ from the MEKK2 protein set forth as SEQ ID NO:2 by no greater than 1, 2 or 3 insertions, deletions, substitutions, or combination thereof, and retain the biological activity of the MEKK2 protein set forth as SEQ ID NO:2. Methods for preparing such variants will be known to one of ordinary skill in the art. The activity of a functionally equivalent variant can be determined using any one of the methods provided herein for determining the activity of the MEKK2 protein set forth as SEQ ID NO:2. Such variants are useful, inter alia, in assays for identification of compounds which bind and/or regulate the MEKK2 protein of the invention.

As used herein, the term “nucleic acid molecule” is intended to include DNA molecules (e.g., cDNA or genomic DNA) and RNA molecules (e.g., mRNA). The nucleic acid molecule may be single-stranded or double-stranded, but preferably is double-stranded DNA.

An used herein, an “isolated nucleic acid molecule” refers to a nucleic acid molecule that is free of gene sequences which naturally flank the nucleic acid in the genomic DNA of the organism from which the nucleic acid is derived (i.e., genetic sequences that are located adjacent to the gene for the isolated nucleic molecule in the genomic DNA of the organism from which the nucleic acid is derived). For example, in various embodiments, an isolated human MEKK2 nucleic acid molecule typically contains less than about 10 kb of nucleotide sequences which naturally flank the nucleic acid molecule in genomic DNA of the cell from which the nucleic acid is derived, and more preferably contains less than about 5, kb, 4 kb, 3 kb, 2 kb, 1 kb, 0.5 kb or 0.1 kb of naturally flanking nucleotide sequences. An “isolated” human MEKK2 nucleic acid molecule may, however, be linked to other nucleotide sequences that do not normally flank the human MEKK2 sequences in genomic DNA (e.g., the human MEKK2 nucleotide sequences may be linked to vector sequences). In certain preferred embodiments, an “isolated” nucleic acid molecule, such as a cDNA molecule, also may be free of other cellular material. However, it is not necessary for the human MEKK2 nucleic acid molecule to be free of other cellular material to be considered “isolated” (e.g., a human MEKK2 DNA molecule separated from other mammalian DNA and inserted into a bacterial cell would still be considered to be “isolated”).

As used herein, the term “hybridizes under high stringency conditions” is intended to describe conditions for hybridization and washing under which nucleotide sequences having substantial homology to each other remain stably hybridized to each other. A preferred, non-limiting example of stringent hybridization conditions includes hybridization in 4× sodium chloride/sodium citrate (SSC), at about 65-70° C. (or hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4X SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀ [Na⁺])+0.41(%G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see e.g., Church and Gilbert (1984) Proc. Natl. Acad. Sci. USA 81:1991-1995, (or alternatively 0.2×SSC, 1% SDS).

The term “% identity” as used in the context of nucleotide and amino acid sequences (e.g., when one amino acid sequence is said to be X% identical to another amino acid sequence) refers to the percentage of identical residues shared between the two sequences, when optimally aligned. To determine the percent identity of two nucleotide or amino acid sequences, the sequences are aligned for optimal comparison purposes (e.g., gaps may be introduced in one sequence for optimal alignment with the other sequence). The residues at corresponding positions are then compared and when a position in one sequence is occupied by the same residue as the corresponding position in the other sequence, then the molecules are identical at that position. The percent identity between two sequences, therefore, is a function of the number of identical positions shared by two sequences (i.e., % identity # of identical positions/total # of positions×100).

Computer algorithms known in the art can be used to optimally align and compare two nucleotide or amino acid sequences to define the percent identity between the two sequences. A preferred, non-limiting example of a mathematical algorithim utilized for the comparison of two sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLASTN and BLASTX programs of Altschul, et al. (1990) J. Mol. Biol. 215:403-10. To obtain gapped alignments for comparison purposes, Gapped BLAST can be utilized as described in Altschul et al., (1997) Nucleic Acids Research 25(17):3389-3402. When utilizing BLAST and Gapped BLAST programs, the default parameters of the respective programs (e.g., BLASTN and BLASTX) can be used. Another preferred, non-limiting example of a mathematical algorithim utilized for the comparison of sequences is William Pearson's LALIGN program. The LALIGN program implements the algorithm of Huang and Miller (1991) Adv. Appl. Math. 12:337-357. When utilizing the LALIGN program for comparing amino acid sequences, a PAM120 weight residue table, a gap length penalty of 12, and a gap penalty of 4 can be used. If multiple programs are used to compare sequences, the program that provides optimal alignment (i.e., the highest percent identity between the two sequences) is used for comparison purposes.

As used herein, a “naturally-occurring” nucleic acid molecule refers to an RNA or DNA molecule having a nucleotide sequence that occurs in nature (e.g., encodes a natural protein).

As used herein, an “antisense” nucleic acid comprises a nucleotide sequence which is complementary to a “sense” nucleic acid encoding a protein, e.g., complementary to the coding strand of a double-stranded cDNA molecule, complementary to an mRNA sequence or complementary to the coding strand of a gene. Accordingly, an antisense nucleic acid can hydrogen bond to a sense nucleic acid.

As used herein, the term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

As used herein, the term “host cell” is intended to refer to a cell into which a nucleic acid of the invention, such as a recombinant expression vector of the invention, has been introduced. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

As used herein, a “transgenic animal” refers to a non-human animal, preferably a mammal, more preferably a mouse, in which one or more of the cells of the animal includes a “transgene”. The term “transgene” refers to exogenous DNA which is integrated into the genome of a cell from which a transgenic animal develops and which remains in the genome of the mature animal, for example directing the expression of an encoded gene product in one or more cell types or tissues of the transgenic animal.

As used herein, a “homologous recombinant animal” refers to a type of transgenic non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

As used herein, an “isolated protein” refers to a protein that is substantially free of other proteins, cellular material and culture medium when isolated from cells or produced by recombinant DNA techniques, or chemical precursors or other chemicals when chemically synthesized.

In one embodiment, a MEKK2 protein is identified based on the presence of at least a “kinase domain” or “catalytic domain” in the protein or corresponding nucleic acid molecule. As used herein, the term “kinase domain” or “catalytic domain” refers to a protein domain consisting of at least about 150-400, preferably about 200-350, more preferably about 220-300, even more preferably at least about 240-280, and even more preferably about 260-261 amino acid residues in length. In one embodiment, a MEKK2 catalytic domain contains at least a “protein kinase ATP-binding region signature” and a “serine/threonine protein kinase active-site signature” (or contains essentially all, i.e., all but one, of the residues of the signature). A “protein kinase ATP-binding region signature” has the consensus sequence [LIV]-G-{P}-G-{P}-[FYWMGSTNH]-[SGA]-{PW}-[LIVCAT]-{PD}-x-[GSTACLIVMFY]-x(5,18)-[LIVMFYWCSTAR]-[AIVP]-[LIVMFAGCKR]-K, corresponding to SEQ ID NO:11. The “protein kinase ATP-binding region signature” includes a glycine-rich stretch of residues in the vicinity of a lysine residue which has been shown to be involved in ATP binding. The glycine-rich stretch, GXGXXG (SEQ ID NO:33) is alternatively referred to as a “G box anchoring domain”. The glycine-rich stretch or “G box anchoring domain” serves as a phosphate anchor, forming bonds to the phosphates of ATP. A “serine/threonine protein kinase active-site signature” has the consensus sequence [LIVMFYC]-x-[HY]-x-D-[LIVMFY]-K-x(2)-N-[LIVMFYCT](3), corresponding to SEQ ID NO: 12. The “serine/threonine protein kinase active-site signature” includes a conserved aspartic acid residue which is important for the catalytic activity of the enzyme. In another embodiment, a MEKK2 catalytic domain is identified based in its ability to retain a functional activity of a MEKK2 protein, particularly a MEKK2 protein (e.g., retains the ability to phosphorylate a MEKK2 substrate) even in the absence of a MEKK2 regulatory domain, as defined herein.

Preferred MEKK2 proteins of the invention comprise a MEKK2 “kinase domain” or “catalytic domain”, as defined herein. Bioactive fragments consisting essentially of (or consisting of) a MEKK2 “kinase domain” or “catalytic domain” are also preferred, for example, fragments consisting essentially of (or consisting of) amino acid residues 356-616 of SEQ ID NO:2. Also preferred are fusion proteins comprising at least a MEKK2 “kinase domain” or “catalytic domain”, as defined herein, for example, comprising a MEKK2 “kinase domain” or “catalytic domain”, operatively linked to a non-MEKK2 polypeptide or protein.

The consensus sequences are described according to standard Prosite Signature designation (e.g., all amino acids are indicated according to their universal single letter designation; X designates any amino acid; X(n) designates any n amino acids, e.g., X (2) designates any 2 amino acids; [LIVM] indicates any one of the amino acids appearing within the brackets, e.g., any one of L, I, V, or M, in the alternative, any one of Leu, Ile, Val, or Met, and {P}indicates any amino acid but the amino acid indicates, e.g., any amino acid but proline).

In another embodiment, a MEKK2 protein is identified based on the presence of at least a “regulatory domain” in the protein or corresponding nucleic acid molecule. As used herein, the term “regulatory domain” refers to a protein domain consisting of at least about 250-500, preferably about 300-450, more preferably about 320-400, even more preferably at least about 340-380, and even more preferably about 360 amino acid residues in length, of which at least 10%, preferably about 15%, and more preferably about 20% of the amino acid residues are serine and/or threonone residues. In another embodiment, a MEKK2 regulatory domain is identified based on its ability to regulate the activity of a MEKK2 catalytic domain. In one exemplary embodiment, a MEKK2 regulatory domain is capable of binding a MEKK2 binding partner such that the activity of a MEKK2 protein is modulated.

As used interchangeably herein, a “MEKK2 activity”, “functional activity of MEKK2”, or “biological activity of MEKK2”, refers to an activity exerted by a MEKK2 protein, polypeptide or nucleic acid molecule as determined in vivo, or in vitro, according to standard techniques. In one embodiment, a MEKK2 activity is a direct activity, such as an association with a MEKK2-target molecule. As used herein, a “target molecule” is a molecule with which a MEKK2 protein binds or interacts in nature, such that MEKK2-mediated function is achieved. As used herein, a “MEKK2” substrate is a molecule with which a MEKK2 protein interacts in vivo or in vitro such that the MEKK2 substrate is phosphorylated by the enzymatic activity (i.e., kinase activity) of the MEKK2 protein. Also as used herein, a MEKK2 “binding partner” is a molecule with which a MEKK2 protein interacts in vivo or in vitro such that the enzymatic activity of the MEKK2 protein is effected. Alternatively, a MEKK2 activity is an indirect activity, such as an activity mediated by interaction of the MEKK2 protein with a MEKK2 target molecule such that the target molecule modulates a downstream cellular activity (e.g., MAPK activity).

In a preferred embodiment, a MEKK2 activity is at least one or more of the following activities: (i) interaction of a MEKK2 protein with a MEKK2 binding partner, wherein the binding partner effects the activity of the MEKK2 molecule; (ii) interaction of a MEKK2 protein with a MEKK2 target molecule, wherein the MEKK2 protein effects the activity of the target molecule; (iii) phosphorylation of a MEKK2 target molecule (e.g., a MAP2K selected from the group consisting of MKK1 (also known as MEK1), MKK2 (also known as MEK2), MKK3, MKK4 (also known as JNKK1 or SEK), MKK5 (also known as MEK5), MKK6, and MKK7 (also known as JNKK2); (iv) autophosphorylation; (v) phosphorylation of a non-target protein, e.g., myelin basic protein (MBP); (vi) mediation of activation of MAPK signal transduction molecules (e.g., the ERKs, for example, ERKs1/2 (also known as p42/p44MAPK) or ERK5 (also known as BMK5), the JNKs, SAPKs and/or p38); (vii) modulation of the activity of a nuclear transcription factor (e.g., an ERK-, JNK- or p38-dependent nuclear transcription factor, for example, ATF 2 or NK-κB); (viii) modulation of ERK-, JNK- or p38-dependent gene transcription (e.g., AP-1 or IL-2 gene transcription); (ix) modulation of cytokine gene expression; and (x) modulation of cellular proliferation, differentiation and/or apoptosis.

Accordingly, another embodiment of the invention features isolated MEKK2 proteins and polypeptides having a MEKK2 activity. Preferred proteins are MEKK2 proteins having at least a MEKK2 catalytic domain and, preferably, a MEKK2 activity. Additional preferred proteins are MEKK2 proteins having at least a MEKK2 regulatory domain and, preferably, a MEKK2 activity. In another preferred embodiment, the isolated protein is a MEKK2 protein having a MEKK2 catalytic domain, a MEKK2 regulatory domain, and a MEKK2 activity.

As used herein, the term “antibody” is intended to include immunoglobulin molecules and immunologically active portions of immunoglobulin molecules, i.e., molecules that contain an antigen binding site which specifically binds (immunoreacts with) an antigen, such as Fab and F(ab′)₂ fragments. The terms “monoclonal antibodies” and “monoclonal antibody composition”, as used herein, refer to a population of antibody molecules that contain only one species of an antigen binding site capable of immunoreacting with a particular epitope of an antigen, whereas the term “polyclonal antibodies” and “polyclonal antibody composition” refer to a population of antibody molecules that contain multiple species of antigen binding sites capable of interacting with a particular antigen. A monoclonal antibody compositions thus typically display a single binding affinity for a particular antigen with which it immunoreacts.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid molecule and the amino acid sequence encoded by that nucleic acid molecule, as defined by the genetic code.

GENETIC CODE

| | | |
|---|---|---|
| Alanine | (Ala, A) | GCA, GCC, GCG, GCT |
| Arginine | (Arg, R) | AGA, ACG, CGA, CGC, CGG, CGT |
| Asparagine | (Asn, N) | AAC, AAT |
| Aspartic acid | (Asp, D) | GAC, GAT |
| Cysteine | (Cys, C) | TGC, TGT |
| Glutamic acid | (Glu, E) | GAA, GAG |
| Glutamine | (Gln, Q) | CAA, CAG |
| Glycine | (Gly, G) | GGA, GGC, GGG, GGT |
| Histidine | (His, H) | CAC, CAT |
| Isoleucine | (Ile, I) | ATA, ATC, ATT |
| Leucine | (Leu, L) | CTA, CTC, CTG, CTT, TTA, TTG |
| Lysine | (Lys, K) | AAA, AAG |
| Methionine | (Met, M) | ATG |
| Phenylalanine | (Phe, F) | TTC, TTT |
| Proline | (Pro, P) | CCA, CCC, CCG, CCT |
| Serine | (Ser, S) | AGC, AGT, TCA, TCC, TCG, TCT |
| Threonine | (Thr, T) | ACA, ACC, ACG, ACT |
| Tryptophan | (Trp, W) | TGG |
| Tyrosine | (Tyr, Y) | TAC, TAT |
| Valine | (Val, V) | GTA, GTC, GTG, GTT |
| Termination | (end) | TAA, TAG, TGA |
| signal | | |

An important and well known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA molecule coding for a human MEKK2 protein of the invention (or any portion thereof) can be used to derive the human MEKK2 amino acid sequence, using the genetic code to translate the DNA or RNA molecule into an amino acid sequence. Likewise, for any human MEKK2-amino acid sequence, corresponding nucleotide sequences that can encode the human MEKK2 protein can be deduced from the genetic code (which, because of its redundancy, will produce multiple nucleic acid sequences for any given amino acid sequence). Thus, description and/or disclosure herein of a human MEKK2 nucleotide sequence should be considered to also include description and/or disclosure of the amino acid sequence encoded by the nucleotide sequence. Similarly, description and/or disclosure of a human MEKK2 amino acid sequence herein should be considered to also include description and/or disclosure of all possible nucleotide sequences that can encode the amino acid sequence.

The human MEKK2 cDNA, which is approximately 1870 nucleotides in length (with nucleotides 1-69 corresponding to 5′ untranslated region (UTR) and nucleotides 70-1870 corresponding to the open reading frame (ORF)) encodes a protein which is approximately 619 amino acid residues in length. The human MEKK2 protein has at least a catalytic domain. A catalytic domain includes, for example, about amino acids 356-616 of SEQ ID NO:2. The human MEKK2 protein further has at least a regulatory domain. A regulatory domain includes, for example, about amino acids 1-355 of SEQ ID NO:2.

Various aspects of the invention are described in further detail in the following subsections:

I. Isolated Nucleic Acid Molecules

One aspect of the invention pertains to isolated nucleic acid molecules that encode human MEKK2. The nucleotide sequence of human MEKK2, and corresponding predicted amino acid sequence, are shown in SEQ ID NOs:1 and 2, respectively. The coding sequence of human MEKK2 is set forth as SEQ ID NO:3. In a preferred embodiment, the nucleic acid molecule comprises the nucleotide sequence of SEQ ID NO:3. In another preferred embodiment, the nucleic acid molecule comprises the nucleotide sequence of SEQ ID NO: 1. In other embodiments, the nucleic acid molecule has at least 99.6% identity, more preferably at least 99.7% identity, even more preferably at least 99.8% identity, and even more preferably at least 99.9% or more identity with the nucleotide sequence of SEQ ID NO:3. In another embodiment, the nucleic acid molecule comprises nucleotides 1137-1917 of SEQ ID NO:3. In yet another embodiment, the nucleic acid molecule has at least 99.6% identity, more preferably at least 99.7% identity, even more preferably at least 99.8% identity, and even more preferably at least 99.9% or more identity with nucleotides 1137-1917 of SEQ ID NO:3.

Nucleic acid molecules that differ from SEQ ID NO:3 due to degeneracy of the genetic code, and thus encode the same human MEKK2 protein as that encoded by SEQ ID NO:1, are encompassed by the invention. Accordingly, in another embodiment, an isolated nucleic acid molecule of the invention has a nucleotide sequence encoding a protein having an amino acid sequence shown in SEQ ID NO:2.

A nucleic acid molecule having the nucleotide sequence of human MEKK2 can be isolated using standard molecular biology techniques and the sequence information provided herein. For example, a human MEKK2 DNA can be isolated from a human genomic DNA library using all or portion of SEQ ID NO:1 as a hybridization probe and standard hybridization techniques (e.g., as described in Sambrook, J., et al. Molecular Cloning: A Laboratory Manual. 2nd, ed., Cold Spring Harbor Laboratory, Cold Spring Harbor, N.Y., 1989). Moreover, a nucleic acid molecule encompassing all or a portion of SEQ ID NO:1 can be isolated by the polymerase chain reaction using oligonucleotide primers designed based upon the sequence of SEQ ID NO:1. Synthetic oligonucleotide primers for PCR amplification can be designed based upon the nucleotide sequence shown in SEQ ID NO:1. A nucleic acid of the invention can be amplified using cDNA or, alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to a human MEKK2 nucleotide sequence can be prepared by standard synthetic techniques, e.g., using an automated DNA synthesizer.

The skilled artisan will further appreciate that minor changes may be introduced by mutation into the nucleotide sequence of SEQ ID NO:1, thereby leading to changes in the amino acid sequence of the encoded protein, without altering the functional activity of the human MEKK2 protein. For example, nucleotide substitutions leading to amino acid substitutions at “non-essential” amino acid residues may be made in the sequence of SEQ ID NO:1. A “non-essential” amino acid residue is a residue that can be altered from the wild-type sequence of human MEKK2 (e.g., the sequence of SEQ ID NO: 2) without altering the functional activity of MEKK2, whereas an “essential” amino acid residue is required for functional activity.

Accordingly, another aspect of the invention pertains to nucleic acid molecules encoding human MEKK2 proteins that contain changes in amino acid residues that are not essential for human MEKK2 activity. Such human MEKK2 proteins differ in amino acid sequence from SEQ ID NO:2 yet retain human MEKK2 activity, i.e., functional variants. These variants preferably, encode at least 1, 2, 3, 4, 5, 6, 7, 8, 9, 10, 11, 12, 13 or more amino acid residues that are unique to the human MEKK2 set forth as SEQ ID NO:2 (i.e., that are not present in other MEKK2 isoforms as set forth in FIGS. 3A-C).

The skilled artisan will further appreciate that mutations can be introduced into the nucleotide sequence of SEQ ID NO:1, leading to an encoded MEKK2 protein having a modified or altered function or biological activity. In one embodiment, nucleotide substitutions leading to amino acid substitutions in the kinase catalytic domain of the MEKK2 protein set forth as SEQ ID NO:2 can be made in the sequence of SEQ ID NO:1. Such mutations are predicted to alter the kinase activity of the mutant kinase facilitating, for example, detailed analysis of the mechanism of action of the human MEKK2 protein set forth as SEQ ID NO:2. In another embodiment, nucleotide substitutions leading to amino acid substitutions in the ATP binding pocket of the MEKK2 protein set forth as SEQ ID NO:2 can be made in the sequence of SEQ ID NO:1. See e.g., Habelhah et al. (2001) J. Biol. Chem. 276:18090-18095 and Specht and Shokat (2002) Curr. Opin. Cell. Biol. 14:155-159. Such mutations are predicted to alter the substrate affinity and/or substrate specificity of the mutant kinase, thus, facilitating screening assays for novel MEKK2 modulators and/or substrates, respectively.

Accordingly, another aspect of the invention pertains to nucleic acid molecules encoding human MEKK2 proteins that contain changes in amino acid residues that are important for human MEKK2 activity. Such human MEKK2 proteins differ in amino acid sequence from SEQ ID NO:2 and exhibit a modified function or biological activity, i.e., modified functional variants. These modified functional variants preferably, encode at least 1, 2, 3, 4, 5, 6, 7, 8, 9, 10, 11, 12, 13 or more amino acid residues that are unique to the human MEKK2 set forth as SEQ ID NO:2 (i.e., that are not present in other MEKK2 isoforms as set forth in FIGS. 3A-C) but differ in at least 1, 2, 3 or more amino acid residues important for MEKK2 biological activity.

An isolated nucleic acid molecule encoding a variant (e.g., a functional variant or modified functional variant) of the human MEKK2 protein set forth as SEQ ID NO:2 can be created by introducing one or more nucleotide substitutions, additions or deletions into the nucleotide sequence of SEQ ID NO:3 such that one or more amino acid substitutions, additions or deletions are introduced into the encoded protein. Mutations can be introduced into SEQ ID NO: 3 by standard techniques, such as site-directed mutagenesis and PCR-mediated mutagenesis. Preferably, conservative amino acid substitutions are made at one or more non-essential amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in human MEKK2 is preferably replaced with another amino acid residue from the same side chain family.

The functional activity of a variant protein can be determined using assays available in the art for assessing MEKK2 activity. For example, MEKK2 proteins (and variants) can be assayed (e.g., in a kinase cascade assay) for the ability to phosphorylate and/or activate MKK1, MKK2, MKK3, MKK4, MKK5, MKK6, and/or MKK7. MEKK2 proteins (and variants) can also be assayed (e.g., in a kinase cascade assay) for the ability to phosphorylate and/or activate “tagged” MKK1, MKK2, MKK3, MKK4, MKK5, MKK6, and/or MKK7 proteins or, alternatively, assayed for the ability to phosphorylate and/or activate fusion proteins comprising all or a portion (or fragment) of a MKK1, MKK2, MKK3, MKK4, MKK5, MKK6, and/or MKK7 protein. MEKK2-proteins (and variants) can also be assayed for the ability to modulate ERK-, JNK- or p38-dependent activities, e.g., ERK-, JNK- or p38-dependent gene expression. MEKK2 proteins (and variants) can also be assayed for the ability to modulate MEKK-dependent cellular proliferation, differentiation and/or apoptosis. MEKK2 proteins (and variants) can also be assayed (e.g, in a phosphorylation assay) for the ability to phosphorylate a kinase inactive mutants of MKK1, MKK2, MKK3, MKK4, MKK5, MKK6, and MKK7). MEKK2 proteins (and variants) can also be assayed in a MEKK2 autophosphorylation assay. In particular, the activity of a human MEKK2 protein or variant can be assessed by determining the extent to which the protein generates unique phosphorylated forms of the MEKK2 through self-incorporation of phosphate from ATP. Moreover, certain MEKK2 substrates, for example MKK4, further autophosphorylate in response to activation by MEKK2. Such substrates are particularly useful indications of MEKK2 activity due to this further autophosphorylation. MEKK2 assays and autophosphorylation assays are described, for example, in Deacon and Blank (1997) J. Biol. Chem. 272:14489-14496 and Widmann et al. (2001) Biochim. Biophys. Acta 1547:167-173. MEKK2 proteins (or variants) can also be assayed for the ability to regulate heterologous promoters, for example, a promoter containing core IL-6 promoter sequences and a MEF-2C site from the c-jun promoter. MEKK2 proteins (or variants) can also be assayed in a cellular context for activation by a cytokine or growth factor, such as EGF, FGF2, IL-1beta, TNFalpha, by IgE and c-kit Ligand (e.g., in mast cells), or by non-native activators such as histamine, dexamethasone, sorbitol, peroxides and oxidative agents, irradiating UV light, phorbol esters, or lipopolysaccharide (LPS), another known activator. MEKK2 proteins (and variants) can also be assayed for the ability to phosphorylate myelin basic protein (MBP). The skilled artisan will appreciate that certain of the above-described assays are also appropriate for determining the activity of a bioactive fragment of a human MEKK2 protein, for example, a kinase domain- or catalytic domain-containing MEKK2 fragment.

Another aspect of the invention pertains to isolated nucleic acid molecules that are antisense to the coding strand of a human MEKK2 mRNA or gene. An antisense nucleic acid of the invention can be complementary to an entire human MEKK2 coding strand, or to only a portion thereof. In one embodiment, an antisense nucleic acid molecule is antisense to a coding region of the coding strand of a nucleotide sequence encoding the human MEKK2 set forth as SEQ ID NO:2 that is unique to said human MEKK2 (as compared to other human MEKK2s, e.g., those set forth in FIGS. 3A-C). In another embodiment, the antisense nucleic acid molecule is antisense to a noncoding region of the coding strand of a nucleotide sequence encoding human MEKK2 that is unique to the human MEKK2 set forth as SEQ ID NO:2. In preferred embodiments, an antisense molecule of the invention comprises at least 5 contiguous nucleotides of the noncoding strand of SEQ ID NO:1, more preferably at least 10, 15, 20, 30, 35, 40, 45, 50 or more contiguous nucleotides of the noncoding strand of SEQ ID NO: 1. In a particularly preferred embodiment, the antisense molecule is between 8 to 30 nucleotides in length.

Yet another aspect of the invention pertains to isolated nucleic acid molecules encoding human MEKK2 fusion proteins. Such nucleic acid molecules, comprising at least a first nucleotide sequence encoding a human MEKK2 protein, polypeptide or peptide operatively linked to a second nucleotide sequence encoding a non-human MEKK2 protein, polypeptide or peptide, can be prepared by standard recombinant DNA techniques. Human MEKK2 fusion proteins are described in further detail below in subsection III.

II. Recombinant Expression Vectors and Host Cells

Another aspect of the invention pertains to vectors, preferably recombinant expression vectors, containing a nucleic acid encoding human MEKK2 (or a portion or variant thereof). The expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector may depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., human MEKK2 proteins, variant forms of human MEKK2 proteins, human MEKK2 fusion proteins and the like).

The recombinant expression vectors of the invention can be designed for expression of human MEKK2 protein in prokaryotic or eukaryotic cells. For example, human MEKK2 can be expressed in bacterial cells such as E. coli, insect cells (using baculovirus expression vectors), yeast cells or mammalian cells. Suitable host cells are discussed further in Goeddel, Gene Expression Technology. Methods in Enzymology 185, Academic Press, San Diego, Calif. (1990). Alternatively, the recombinant expression vector may be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors can serve one or more purposes: 1) to increase expression of recombinant protein; 2) to increase the solubility of the recombinant protein; 3) to aid in the purification of the recombinant protein by acting as a ligand in affinity purification; 4) to provide an epitope tag to aid in detection and/or purification of the protein; and/or 5) to provide a marker to aid in detection of the protein (e.g., a color marker using β-galactosidase fusions). Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors fuse glutathione S-transferase (GST), maltose E binding protein, protein A, or polyhistidine, to the target recombinant protein. Recombinant proteins also can be expressed in eukaryotic cells as fusion proteins for the same purposes discussed above.

In another embodiment, the human MEKK2 expression vector is a yeast expression vector. Alternatively, human MEKK2 can be expressed in insect cells using baculovirus expression vectors. Baculovirus vectors available for expression of proteins in cultured insect cells (e.g., Sf 9 cells). A preferred human MEKK2 expression vector is the pQE-TriSystem vector that allows for expression of the target recombinant protein as a fusion protein with an N-terminal polyhistidine tag, or N-terminal GST tag. The vector is capable of expressing the fusion protein in bacteria, in baculovirus-infected cells (e.g., Sf9 or Sf21 cells), and in various mammalian cells. Vectors expressing the target recombinant protein with a C-terminal tag are also within the scope of the invention.

In yet another embodiment, a nucleic acid of the invention is expressed in mammalian cells using a mammalian expression vector. Examples of mammalian expression vectors include pMex-NeoI, pCDM8 (Seed, B., (1987) Nature 329:840) and pMT2PC (Kaufman et al. (1987), EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. in another embodiment, the recombinant mammalian expression vector is capable of directing expression of the nucleic acid preferentially in a particular cell type (e.g., tissue-specific regulatory elements are used to express the nucleic acid). Moreover, inducible regulatory systems for use in mammalian cells are known in the art. Accordingly, in another embodiment, the invention provides a recombinant expression vector in which human MEKK2 DNA is operatively linked to an inducible eukaryotic promoter, thereby allowing for inducible expression of human MEKK2 protein in eukaryotic cells.

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to human MEKK2 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. Antisense expression vectors are described, for example, in U.S. Pat. No. 6,287,860 (incorporated herein by reference). Exemplary vectors express RNA molecules which are antisense to the human MEKK2 nucleotide sequence set forth as SEQ ID NO:1 and, preferably, are specific to the human MEKK2 nucleotide sequence set forth as SEQ ID NO:1.

Another aspect of the invention pertains to recombinant host cells into which a vector, preferably a recombinant expression vector, of the invention has been introduced. A host cell may be any prokaryotic or eukaryotic cell. For example, human MEKK2 protein may be expressed in bacterial cells such as E. coli, insect cells, yeast or mammalian cells (such as Chinese hamster ovary cells (CHO) or COS cells). Other suitable host cells are known to those skilled in the art. Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker may be introduced into a host cell on the same vector as that encoding human MEKK2 or may be introduced on a separate vector. Cells stably transfected with the introduced nucleic acid can be identified by drug selection (e.g., cells that have incorporated the selectable marker gene will survive, while the other cells die). A host cell of the invention, such as a prokaryotic or eukaryotic host cell in culture, can be used to produce (i.e., express) human MEKK2 protein. The skilled artisan will appreciate that certain post-translational modifications will result based on the choice of host cell selected for expressing the MEKK2 proteins of the invention. In a preferred embodiment, the MEKK2 protein produced is a phosphorylated MEKK2 protein.

The invention further provides methods for producing human MEKK2 protein using the host cells of the invention. In one embodiment, the method comprises culturing the host cell of invention (into which a recombinant expression vector encoding human MEKK2 has been introduced) in a suitable medium until human MEKK2 is produced. In another embodiment, the method further comprises isolating human MEKK2 from the medium or the host cell. In its native form the human MEKK2 protein is an intracellular protein and, accordingly, recombinant human MEKK2 protein can be expressed intracellularly in a recombinant host cell and then isolated from the host cell, e.g., by lysing the host cell and recovering the recombinant human MEKK2 protein from the lysate. Alternatively, recombinant human MEKK2 protein can be prepared as an extracellular protein by operatively linking a heterologous signal sequence to the amino-terminus of the protein such that the protein is secreted from the host cells. In this case, recombinant human MEKK2 protein can be recovered from the culture medium in which the cells are cultured.

Certain host cells of the invention can also be used to produce nonhuman transgenic animals. For example, in one embodiment, a host cell of the invention is a fertilized oocyte or an embryonic stem cell into which human MEKK2-coding sequences have been introduced. Such host cells can then be used to create non-human transgenic animals in which exogenous human MEKK2 sequences have been introduced into their genome or homologous recombinant animals in which endogenous MEKK2 sequences have been altered. Such animals are useful for studying the function and/or activity of human MEKK2 and for identifying and/or evaluating modulators of human MEKK2 activity. Accordingly, another aspect of the invention pertains to nonhuman transgenic animals which contain cells carrying a transgene encoding a human MEKK2 protein or a portion of a human MEKK2 protein. In another embodiment, the transgenic animal contains cells carrying a transgene that alters an endogenous gene encoding human MEKK2 protein (e.g., homologous recombinant animals in which the endogenous MEKK2 gene has been functionally disrupted or “knocked out”, or the nucleotide sequence of the endogenous MEKK2 gene has been mutated or the transcriptional regulatory region of the endogenous MEKK2 gene has been altered). In addition to the foregoing, the skilled artisan will appreciate that other approaches known in the art for homologous recombination can be applied to the instant invention.

III. Isolated Human MEKK2 Proteins and Anti-Human MEKK2 Antibodies

Another aspect of the invention pertains to isolated human MEKK2 proteins. Preferably, the human MEKK2 protein comprises the amino acid sequence of SEQ ID NO:2. In other embodiments, the protein has at least 99.1% identity, more preferably 99.2% identity, even more preferably 99.3% identity, and even more preferably at least 99.4%, 99.5%, 99.6%, 99.7%, 99.8%, 99.9% or more identity with the amino acid sequence of SEQ ID NO:2.

In other embodiments, the invention provides isolated portions of the human MEKK2 protein. For example, the invention further encompasses an amino-terminal portion of human MEKK2 that includes a regulatory domain. This portion encompasses, for example, about amino acids 1-355 of the amino acid sequence set forth as SEQ ID NO:2. Another isolated portion of human MEKK2 provided by the invention is a carboxy-terminal catalytic domain. This portion encompasses, for example, about amino acids 356-616 of the amino acid sequence set forth as SEQ ID NO:2.

Human MEKK2 proteins of the invention are preferably produced by recombinant DNA techniques. For example, a nucleic acid molecule encoding the protein is cloned into an expression vector (as described above), the expression vector is introduced into a host cell (as described above) and the human MEKK2 protein is expressed in the host cell. The human MEKK2 protein can then be isolated from the cells by an appropriate purification scheme using standard protein purification techniques. Alternative to recombinant expression, a human MEKK2 polypeptide can be synthesized chemically using standard peptide synthesis techniques. Moreover, human MEKK2 protein can be isolated from cells (e.g., from T cells), for example by immunoprecipitation using an anti-human MEKK2 antibody. The skilled artisan will appreciate that the nature and extent of post-translational modification can be influenced by the choice of host cell for expressing recombinant MEKK2 proteins of the invention. Moreover, MEKK2 proteins can be chemically synthesized using modified amino acid residues. This invention contemplates and includes not only the native MEKK2 protein encoded by the nucleotide sequence set forth as SEQ ID NO:1 but also includes modified and/or recombinant proteins comprising modified amino acid residues, in particular, phosphorylated proteins including phosphorylated residues.

The invention also provides human MEKK2 fusion proteins. As used herein, a human MEKK2 “fusion protein” comprises a human MEKK2 polypeptide operatively linked to a polypeptide other than human MEKK2. A “human MEKK2 polypeptide” refers to a polypeptide having an amino acid sequence corresponding to human MEKK2 protein, or a peptide fragment thereof which is unique to the human MEKK2 protein set forth as SEQ ID NO:2, or a variant thereof, whereas a “polypeptide other than human MEKK2” refers to a polypeptide having an amino acid sequence corresponding to another protein. Within the fusion protein, the term “operatively linked” is intended to indicate that the human MEKK2 polypeptide and the other polypeptide are fused in-frame to each other. The other polypeptide may be fused to the N-terminus or C-terminus of the human MEKK2 polypeptide. For example, in one embodiment, the fusion protein is a GST-human MEKK2 fusion protein in which the human MEKK2 sequences are fused to the C-terminus of the GST sequences. In another embodiment, the fusion protein is a HIS6-MEKK2 fusion protein in which the human MEKK2 sequences are fused to the C-terminus of a polyhistidine sequence. In another embodiment, the fusion protein is a flag-taggedMEKK2, a myc-tagged MEKK2 or a HA-tagged MEKK2. Such fusion proteins can facilitate the purification of recombinant human MEKK2.

Preferably, a human MEKK2 fusion protein of the invention is produced by standard recombinant DNA techniques. For example, DNA fragments coding for the different polypeptide sequences are ligated together in-frame in accordance with conventional techniques, for example employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide or an polyhistidine tag). A human MEKK2-encoding nucleic acid can be cloned into such an expression vector such that the fusion moiety is linked in-frame to the human MEKK2 protein.

An isolated human MEKK2 protein, or fragment thereof, can be used as an immunogen to generate antibodies that bind specifically to human MEKK2 using standard techniques for polyclonal and monoclonal antibody preparation. The human MEKK2 protein can be used to generate antibodies or, alternatively, an antigenic peptide fragment of human MEKK2 can be used as the immunogen. An antigenic peptide fragment of human MEKK2 typically comprises at least 8 amino acid residues of the amino acid sequence shown in SEQ ID NO: 2 and encompasses an epitope of human MEKK2 such that an antibody raised against the peptide forms a specific immune complex with human MEKK2. Preferably, the antigenic peptide comprises at least 10 amino acid residues, more preferably at least 15 amino acid residues, even more preferably at least 20 amino acid residues, and most preferably at least 30 amino acid residues. Preferred epitopes encompassed by the antigenic peptide are regions of human MEKK2 that are located on the surface of the protein, e.g., hydrophilic regions, and that are unique to the human MEKK2 sequence set forth as SEQ ID NO:2, as compared to other human MEKK2 proteins or MEKK2 proteins from other species, such as mouse (i.e., an antigenic peptide that spans a region of human MEKK2 that is not conserved across isoforms is used as immunogen; such non-conserved regions/residues are shown in FIGS. 3A-C). A standard hydrophobicity analysis of the human MEKK2 protein can be performed to identify hydrophilic regions.

A human MEKK2 immunogen typically is used to prepare antibodies by immunizing a suitable subject, (e.g., rabbit, goat, mouse or other mammal) with the immunogen. An appropriate immunogenic preparation can contain, for examples, recombinantly expressed human MEKK2 protein or a chemically synthesized human MEKK2 peptide. The preparation can further include an adjuvant, such as Freund's complete or incomplete adjuvant, or similar immunostimulatory agent. Immunization of a suitable subject with an immunogenic human MEKK2 preparation induces a polyclonal anti-human MEKK2 antibody response.

Accordingly, another aspect of the invention pertains to anti-human MEKK2 antibodies. Polyclonal anti-human MEKK2 antibodies can be prepared as described above by immunizing a suitable subject with a human MEKK2 immunogen. The anti-human MEKK2 antibody titer in the immunized subject can be monitored over time by standard techniques, such as with an enzyme linked immunosorbent assay (ELISA) using immobilized human MEKK2. If desired, the antibody molecules directed against human MEKK2 can be isolated from the mammal (e.g., from the blood) and further purified by well known techniques, such as protein A chromatography to obtain the IgG fraction. At an appropriate time after immunization, e.g, when the anti-human MEKK2 antibody titers are highest, antibody-producing cells can be obtained from the subject and used to prepare monoclonal antibodies by standard techniques. The technology for producing monoclonal antibody hybridomas is well known. Briefly, an immortal cell line (typically a myeloma) is fused to lymphocytes (typically splenocytes) from a mammal immunized with a human MEKK2 immunogen as described above, and the culture supernatants of the resulting hybridoma cells are screened to identify a hybridoma producing a monoclonal antibody that binds specifically to human MEKK2.

Any of the many well known protocols used for fusing lymphocytes and immortalized cell lines can be applied for the purpose of generating an anti-human MEKK2 monoclonal antibody. Moreover, the ordinary skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind human MEKK2, e.g., using a standard ELISA assay.

Alternative to preparing monoclonal antibody-secreting hybridomas, a monoclonal anti-human MEKK2 antibody can be identified and isolated by screening a recombinant combinatorial immunoglobulin library (e.g., an antibody phage display library) with human MEKK2 to thereby isolate immunoglobulin library members that bind human MEKK2.

Additionally, recombinant anti-human MEKK2 antibodies, such as chimeric and humanized monoclonal antibodies, comprising both human and non-human portions, which can be made using standard recombinant DNA techniques, are within the scope of the invention.

A preferred anti-human MEKK2 antibody of the instant invention is directed against portions of MEKK2 that are unique to the MEKK2 protein set forth as SEQ ID NO:2 (as compared to MEKK2 isoforms previously described in the art). Another preferred anti-human MEKK2 antibody is directed against a phosphorylated human MEKK2 protein, e.g., and autophosphorylated MEKK2 protein generated according the methods described in Example 4.

An anti-human MEKK2 antibody (e.g., monoclonal antibody) can be used to isolate human MEKK2 by standard techniques, such as affinity chromatography or immunoprecipitation. An anti-human MEKK2 antibody can facilitate the purification of natural human MEKK2 from cells and of recombinantly produced human MEKK2 expressed in host cells. Moreover, an anti-human MEKK2 antibody can be used to detect human MEKK2 protein (e.g., in a cellular lysate or cell supernatant). Detection may be facilitated by coupling (i.e., physically linking) the antibody to a detectable substance. Accordingly, in one embodiment, an anti-human MEKK2 antibody of the invention is labeled with a detectable substance. Examples of detectable substances include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ¹²⁵I, ¹³¹I, ³⁵S or ³H.

Yet another aspect of the invention pertains to anti-human MEKK2 antibodies that are obtainable by a process comprising:

(a) immunizing an animal with an immunogenic human MEKK2 protein, or an immunogenic portion thereof unique to human MEKK2 protein; and

(b) isolating from the animal antibodies that specifically bind to a human MEKK2 protein.

Methods for immunization and recovery of the specific anti-human MEKK2 antibodies are described further above.

IV. Pharmaceutical Compositions

Human MEKK2 modulators of the invention (e.g., human MEKK2 inhibitory or stimulatory agents, including human MEKK2 proteins and antibodies) can be incorporated into pharmaceutical compositions suitable for administration. Such compositions typically comprise the modulatory agent and a pharmaceutically acceptable carrier. As used herein the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

In one embodiment, the active compounds are prepared with carriers that will protect the compound against rapid elimination from the body, such as a controlled release formulation. Methods for preparation of such formulations will be apparent to those skilled in the art. Liposomal suspensions (including liposomes targeted to infected cells with monoclonal antibodies to viral antigens) can also be used as pharmaceutically acceptable carriers.

V. Methods of the Invention

Another aspect of the invention pertains to methods of using the various human MEKK2 compositions of the invention. For example, the invention provides a method for detecting the presence of human MEKK2 activity in a biological sample. The method involves contacting the biological sample with an agent capable of detecting human MEKK2 activity, such as human MEKK2 protein or human MEKK2 mRNA, such that the presence of human MEKK2 activity is detected in the biological sample.

A preferred agent for detecting human MEKK2 mRNA is a labeled nucleic acid probe capable of specifically hybridizing to a human MEKK2 mRNA corresponding to SEQ ID NO:1. The nucleic acid probe can be, for example, the human MEKK2 DNA of SEQ ID NO: 1, or a portion thereof unique to human MEKK2 (as compared to other human MEKK2 isoforms or to MEKK2 from other species, such as mouse), for example, an oligonucleotide of at least 5, 10, 15, 20, 25, 30, 35, 40, 45 50 or more nucleotides in length and sufficient to specifically hybridize under stringent conditions to the human MEKK2 mRNA.

A preferred agent for detecting human MEKK2 protein is a labeled antibody capable of specifically binding to the human MEKK2 protein set forth as SEQ ID NO:2. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids. For example, techniques for detection of human MEKK2 mRNA include Northern hybridizations and in situ hybridizations. Techniques for detection of human MEKK2 protein include enzyme linked immunosorbent assays (ELISAs), Western blots, immunoprecipitations and immunofluorescence.

The invention further provides methods for identifying modulators, i.e., candidate or test compounds or agents (e.g., peptides, peptidomimetics, small molecules or other drugs) which bind to human MEKK2 proteins, have a stimulatory or inhibitory effect on, for example, human MEKK2 expression or activity, or have a stimulatory or inhibitory effect on, for example, the expression or activity of another component in the MEKK2 signaling cascade. Such methods are also referred to herein as a “screening assays”.

In one embodiment, the invention provides assays to screen for candidate or test compounds which modulate expression of a human MEKK2 protein, or biologically active fragment thereof. In another embodiment, the invention provides assays to screen for candidate or test compounds which bind to or modulate the activity of a human MEKK2 protein, or biologically active fragment thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. Libraries of compounds may be presented in solution, or on beads, chips, bacteria, spores, plasmids, or phage.

The invention provides both cell-free and cell-based screening assays. In one embodiment, an assay of the present invention is a cell-free assay in which a human MEKK2 protein, or biologically active fragment thereof, is contacted with a test compound and the ability of the test compound to bind to the MEKK2 protein, or biologically active fragment thereof, is determined. Preferred biologically active fragments of the MEKK2 proteins to be used in assays of the present invention include fragments having at least one biological activity of the intact MEKK2 protein, as described herein. Binding of the test compound to the MEKK2 protein can be determined either directly or indirectly. In a preferred embodiment, the assay includes contacting the MEKK2 protein, or biologically active fragment thereof, with a known compound which binds MEKK2 to form an assay mixture, contacting the assay mixture with a test compound, and determining the ability of the test compound to interact with a MEKK2 protein, wherein determining the ability of the test compound to interact with the MEKK2 protein comprises determining the ability of the test compound to preferentially bind to MEKK2, or biologically active fragment thereof, as compared to the known compound.

In another embodiment, the assay is a cell-free assay in which a MEKK2 protein, or biologically active fragment thereof, is contacted with a test compound and the ability of the test compound to modulate (e.g., stimulate or inhibit) the activity of the MEKK2 protein, or biologically active fragment thereof, is determined. Determining the ability of the test compound to modulate the activity of the MEKK2 protein can be accomplished, for example, by determining a MEKK2 activity in the presence of the test compound and comparing that to the MEKK2 activity in the absence of the test compound (or comparing to any other suitable control, for example, a known or normalized control value, negative control, e.g., a buffer or solvent control, etc). In yet another embodiment, the cell-free assay involves contacting a MEKK2 protein, or biologically active fragment thereof, with a known compound which binds to or activates the MEKK2 protein to form an assay mixture, contacting the assay mixture with a test compound, and determining the ability of the test compound to modulate a MEKK2 activity.

The invention further provides methods (e.g., screening assays) for identifying compounds that modulate the activity of a human MEKK2 protein, as follows:

In one embodiment, the invention provides a method for identifying a compound that modulates the activity of a human MEKK2 protein, comprising

providing an indicator composition that comprises a human MEKK2 protein;

contacting the indicator composition with a test compound; and

determining the effect of the test compound on the activity of the human MEKK2 protein in the indicator composition to thereby identify a compound that modulates the activity of a human MEKK2 protein.

In a preferred embodiment of the screening assays of the invention, the indicator composition comprises an indicator cell, wherein said indicator cell comprises: (i) the a human MEKK2 protein and (ii) a reporter gene responsive to the human MEKK2 protein. Preferably, the indicator cell contains:

i) a recombinant expression vector encoding the human MEKK2; and

ii) a vector comprising regulatory sequences of an ATF 2-responsive gene operatively linked a reporter gene; and said method comprises:

a) contacting the indicator cell with a test compound;

b) determining the level of expression of the reporter gene in the indicator cell in the presence of the test compound; and

c) comparing the level of expression of the reporter gene in the indicator cell in the presence of the test compound with the level of expression of the reporter gene in the indicator cell in the absence of the test compound to thereby identify a compound that modulates the activity of human MEKK2.

In another preferred embodiment, the indicator composition comprises a preparation of: (i) a human MEKK2 protein and (ii) a DNA molecule to which an ATF 2 transcription factor binds, and said method comprises:

a) contacting the indicator composition with a test compound;

b) determining the degree of interaction of an ATF 2 transcription factor and the DNA molecule in the presence of the test compound; and

c) comparing the degree of interaction of ATF 2 transcription factor and the DNA molecule in the presence of the test compound with the degree of interaction of the ATF 2 transcription factor and the DNA molecule in the absence of the test compound to thereby identify a compound that modulates the activity of human MEKK2.

In another preferred embodiment, the method identifies proteins that interact with human MEKK2. In this embodiment,

the indicator composition is an indicator cell, which indicator cell comprises:

i) a reporter gene operably linked to a transcriptional regulatory sequence; and

ii) a first chimeric gene which encodes a first fusion protein, said first fusion protein including human MEKK2;

the test compound comprises a library of second chimeric genes, which library encodes second fusion proteins;

expression of the reporter gene being sensitive to interactions between the first fusion protein, the second fusion protein and the transcriptional regulatory sequence; and

wherein the effect of the test compound on human MEKK2 in the indicator composition is determined by determining the level of expression of the reporter gene in the indicator cell to thereby identify a test compound comprising a protein that interacts with human MEKK2.

Recombinant expression vectors that can be used for expression of human MEKK2 in the indicator cell are known in the art (see discussions above). In one embodiment, within the expression vector the human MEKK2-coding sequences are operatively linked to regulatory sequences that allow for constitutive expression of human MEKK2 in the indicator cell (e.g., viral regulatory sequences, such as a cytomegalovirus promoter/enhancer, can be used). Use of a recombinant expression vector that allows for constitutive expression of human MEKK2 in the indicator cell is preferred for identification of compounds that enhance or inhibit the activity of human MEKK2. In an alternative embodiment, within the expression vector the human MEKK2-coding sequences are operatively linked to regulatory sequences of the endogenous human MEKK2 gene (i.e., the promoter regulatory region derived from the endogenous human MEKK2 gene). Use of a recombinant expression vector in which human MEKK2 expression is controlled by the endogenous regulatory sequences is preferred for identification of compounds that enhance or inhibit the transcriptional expression of human MEKK2.

A variety of reporter genes are known in the art and are suitable for use in the screening assays of the invention. Examples of suitable reporter genes include those which encode chloramphenicol acetyltransferase, beta-galactosidase, alkaline phosphatase or luciferase. Standard methods for measuring the activity of these gene products are known in the art. Likewise, a variety of cell types are suitable for use as an indicator cell in the screening assay. Preferably a cell line is used which does not normally express human MEKK2. Mammalian cell lines as well as yeast cells can be used as indicator cells.

In one embodiment, the level of expression of the reporter gene in the indicator cell in the presence of the test compound is higher than the level of expression of the reporter gene in the indicator cell in the absence of the test compound and the test compound is identified as a compound that stimulates the expression or activity of human MEKK2. In another embodiment, the level of expression of the reporter gene in the indicator cell in the presence of the test compound is lower than the level of expression of the reporter gene in the indicator cell in the absence of the test compound and the test compound is identified as a compound that inhibits the expression or activity of human MEKK2.

Alternative to the use of a reporter gene construct, compounds that modulate the expression or activity of human MEKK2 can be identified by using other “read-outs.” For example, an indicator cell can be transfected with a human MEKK2 expression vector, incubated in the presence and in the absence of a test compound, and MEKK2 activity be assessed by detecting the mRNA of an ATF 2-responsive gene product. Standard methods-for detecting mRNA, such as reverse transcription-polymerase chain reaction (RT-PCR) are known in the art. Alternatively, MEKK2 activity can be assessed by detecting ATF2 mRNA levels.

As described above, the invention provides a screening assay for identifying compounds that modulate the activity of human MEKK2 by assessing the interaction between ATF 2 and a regulatory element of an ATF 2-responsive gene. Assays are known in the art that detect the interaction of a DNA binding protein with a target DNA sequence (e.g., electrophoretic mobility shift assays, DNAse I footprinting assays and the like). By performing such assays in the presence and absence of test compounds, these assays can be used to identify compounds that modulate (e.g., inhibit or enhance) the interaction of the DNA binding protein with its target DNA sequence.

In one embodiment, the amount of binding of ATF 2 to the DNA fragment in the presence of the test compound is greater than the amount of binding of ATF 2 to the DNA fragment in the absence of the test compound, in which case the test compound is identified as a compound that enhances activity of human MEKK2. In another embodiment, the amount of binding of ATF 2 to the DNA fragment in the presence of the test compound is less than the amount of binding of ATF 2 to the DNA fragment in the absence of the test compound, in which case the test compound is identified as a compound that inhibits activity of human MEKK2.

In any of the above assay formats featuring a MEKK2-responsive transcription factor, NF-κB can be substituted for ATF 2.

Yet another aspect of the invention pertains to methods wherein a human MEKK2 protein or cell expressing a human MEKK2 protein is utilized in an assay designed specific modulators, i.e., a specificity assay. In one embodiment, a MEKK2 protein of the invention is used in an assay to identify a MEKK2-specific modulator. For example, a compound identified in screening assay formatted to identify a MEKK2 modulator can be screened as well in an assay formatted to identify a MEKK1, MEKK2 or MEKK3 modulator. Compounds active in the former and inactive in the latter are referred to as MEKK2-specific modulators. In another example, a MEKK2 protein of the invention is used in an assay to identify a MEKK2-specific modulator (e.g., as compared to another, non-MEKK2 kinase). For example, a compound identified in screening assay formatted to identify a MEKK2 modulator can be screened as well in an assay formatted to identify modulators of a non-MEKK2 kinase. Compounds active in the former and inactive in the latter are also referred to as MEKK2-specific modulators.

In another embodiment, a MEKK2 protein of the invention is used to identify a modulator specific for MEKK1, MEKK3 or MEKK4. For example, a compound identified in screening assay formatted to identify a modulator of MEKK1, MEKK3 or MEKK4 can be screened as well in an assay formatted to identify a MEKK2 modulator. Compounds active in the former and inactive in the latter are referred to as MEKK1-, MEKK3 or MEKK4-specific modulators, respectively.

Other embodiments contemplated by the instant inventors include kits for performing the screening assays described herein. In particular, kits comprising recombinant human MEKK2, MEKK2 fusion proteins, MEKK2-specific antibodies and/or cells expressing human MEKK2 are featured. Preferred kits include instructions for use.

In more than one embodiment of the above assay methods of the present invention, it may be desirable to immobilize either one or more assay components to facilitate separation of complexed from uncomplexed forms of one or more reagents, as well as to accommodate automation of the assay, e.g., a scintillation proximity assay (SPA) or enzyme-linked immunsorbent (ELISA) assay. Screening assays can be carried out in any vessel suitable for containing the components, e.g., microtiter plates, test tubes, and micro-centrifuge tubes. In one embodiment, a fusion protein can be provided which adds a domain that allows one or more components to be bound to a matrix. For example, glutathione-S-transferase/MEKK2 fusion proteins or glutathione-S-transferase/target fusion proteins can be adsorbed onto glutathione sepharose beads (Sigma Chemical, St. Louis, Mo.) or glutathione derivatized micrometer plates, which are then combined with the test compound or the test compound and either the non-adsorbed target protein or MEKK2 protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of MEKK2 binding or activity determined using standard techniques.

Other techniques for immobilizing proteins on matrices can also be used in the screening assays of the invention. For example, either a MEKK2 protein or a MEKK2 substrate or target molecule can be immobilized utilizing conjugation of biotin and streptavidin. Biotinylated MEKK2 protein, substrates, or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with MEKK2 protein or target molecules but which do not interfere with binding of the MEKK2 protein to its target molecule can be derivatized to the wells of the plate, and unbound target or MEKK2 protein trapped in the wells by antibody conjugation. Methods for detecting such complexes, in addition to those described above for the GST-immobilized complexes, include immunodetection of complexes using antibodies reactive with the MEKK2 protein or target molecule, as well as enzyme-linked assays which rely on detecting an enzymatic activity associated with the MEKK2 protein or target molecule.

In order to facilitate monitoring of one the activities described herein, one or more assay components (e.g., a human MEKK2 protein or polypeptide, or fragment or portion thereof, or a MEKK2 binding partner or target molecule) can be coupled to a radioisotope or enzymatic label. For example, compounds can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of assay components to interact without the labeling of any of the interactants. For example, a microphysiometer can be used to detect the interaction of a MEKK2 protein with a MEKK2 binding partner or target molecule without the labeling of either the MEKK2 protein or MEKK2 binding partner or target molecule. (McConnell, H. M. et al. (1992) Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between assay components. Determining the binding of assay components can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In another embodiment, an assay is a cell-based assay in which a cell which expresses a human MEKK2 protein or biologically active fragment or portion thereof is contacted with a test compound and the ability of the test compound to modulate MEKK2 activity is determined.

Determining the ability of the test compound to modulate MEKK2 activity can be accomplished by monitoring, for example: (i) interaction of a MEKK2 protein with a MEKK2 binding partner, wherein the binding partner effects the activity of the MEKK2 molecule; (ii) interaction of a MEKK2 protein with a MEKK2 target molecule, wherein the MEKK2 protein effects the activity of the target molecule; (iii) phosphorylation of a MEKK2 target molecule (e.g., a MAP2K selected from the group consisting of MKK1 (also known as MEK1), MKK2 (also known as MEK2), MKK3, MKK4 (also known as JNKK1 or SEK), MKK5 (also known as MEK5), MKK6, and MKK7 (also known as JNKK2); (iv) autophosphorylation (v) phosphorylation of a non-target protein, e.g., myelin basic protein (MBP); (vi) mediation of activation of MAPK signal transduction molecules (e.g., the ERKs, for example, ERKs1/2 (also known as p42/p44MAPK) or ERK5 (also known as BMK5), the JNKs, SAPKs and/or p38); (vii) modulation of the activity of a nuclear transcription factor (e.g., an ERK-, JNK- or p38-dependent nuclear transcription factor, for example, ATF 2 or NK-κB); (viii) modulation of ERK-, JNK- or p38-dependent gene transcription (e.g., AP-1 or IL-2 gene transcription); (ix) modulation of cytokine gene expression; and (x) modulation of cellular proliferation, differentiation and/or apoptosis.

In another embodiment, modulators of MEKK2 expression are identified in a method wherein a cell is contacted with a candidate compound and the expression of MEKK2 mRNA or protein in the cell is determined. The level of expression of MEKK2 mRNA or protein in the presence of the candidate compound is compared to the level of expression of MEKK2 mRNA or protein in the absence of the candidate compound. The candidate compound can then be identified as a modulator of MEKK2 expression based on this comparison. For example, when expression of MEKK2 mRNA or protein is greater (statistically significantly greater) in the presence of the candidate compound than in its absence, the candidate compound is identified as a stimulator of MEKK2 mRNA or protein expression. Alternatively, when expression of MEKK2 mRNA or protein is less (statistically significantly less) in the presence of the candidate compound than in its absence, the candidate compound is identified as an inhibitor of MEKK2 mRNA or protein expression. The level of MEKK2 mRNA or protein expression in the cells can be determined by methods described herein for detecting MEKK2 mRNA or protein.

In another aspect, the invention pertains to a combination of two or more of the assays described herein. For example, a modulating agent can be identified using a cell-based or a cell-free assay.

This invention further pertains to novel agents identified by the above-described screening assays. Accordingly, it is within the scope of this invention to further use an agent identified as described herein in an appropriate animal model. For example, an agent identified as described herein (e.g., a MEKK2 modulating agent, an antisense MEKK2 nucleic acid molecule, a MEKK2-specific antibody, or a MEKK2 binding partner or target molecule) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

Yet another aspect of the invention pertains to methods of modulating human MEKK2 activity in a cell. The modulatory methods of the invention involve contacting the cell with an agent that modulates human MEKK2 activity such that human MEKK2 activity in the cell is modulated. The agent may act by modulating the activity of human MEKK2 protein in the cell or by modulating transcription of the human MEKK2 gene or translation of the human MEKK2 mRNA. As used herein, the term “modulating” is intended to include inhibiting or decreasing human MEKK2 activity and stimulating or increasing human MEKK2 activity. Accordingly, in one embodiment, the agent inhibits human MEKK2 activity. In another embodiment, the agent stimulates human MEKK2 activity.

A. Inhibitory Agents

According to a modulatory method of the invention, human MEKK2 activity is inhibited in a cell by contacting the cell with an inhibitory agent. Inhibitory agents of the invention can be, for example, intracellular binding molecules that act to inhibit the expression or activity of human MEKK2. As used herein, the term “intracellular binding molecule” is intended to include molecules that act intracellularly to inhibit the expression or activity of a protein by binding to the protein itself, to a nucleic acid (e.g., an mRNA molecule) that encodes the protein or to a target with which the protein indirectly interacts (e.g., to a DNA target sequence to which ATF 2 binds). Examples of intracellular binding molecules, described in further detail below, include antisense human MEKK2 nucleic acid molecules (e.g, to inhibit translation of human MEKK2 mRNA), intracellular anti-human MEKK2 antibodies (e.g., to inhibit the activity of human MEKK2 protein) and dominant negative mutants of the human MEKK2 protein.

In one embodiment, an inhibitory agent of the invention is an antisense nucleic acid molecule that is complementary to a gene encoding human MEKK2 or to a portion of said gene, or a recombinant expression vector encoding said antisense nucleic acid molecule. The use of antisense nucleic acids to downregulate the expression of a particular protein in a cell is well known in the art. An antisense nucleic acid for inhibiting the expression of human MEKK2 protein in a cell can be designed based upon the nucleotide sequence encoding the human MEKK2 protein (e.g., SEQ ID NO: 1), constructed according to the rules of Watson and Crick base pairing. An antisense oligonucleotide can be chemically synthesized using naturally occurring nucleotides or variously modified nucleotides designed to increase the biological stability of the molecules or to increase the physical stability of the duplex formed between the antisense and sense nucleic acids, e.g. phosphorothioate derivatives and acridine substituted nucleotides can be used. To inhibit human MEKK2 expression in cells in culture, one or more antisense oligonucleotides can be added to cells in culture media, typically at about 200 μg oligonucleotide/ml.

Alternatively, an antisense nucleic acid can be produced biologically using an expression vector into which a nucleic acid has been subcloned in an antisense orientation (i.e., nucleic acid transcribed from the inserted nucleic acid will be of an antisense orientation to a target nucleic acid of interest). Regulatory sequences operatively linked to a nucleic acid cloned in the antisense orientation can be chosen which direct the expression of the antisense RNA molecule in a cell of interest, for instance promoters and/or enhancers or other regulatory sequences can be chosen which direct constitutive, tissue specific or inducible expression of antisense RNA. The antisense expression vector is prepared as described above for recombinant expression vectors, except that the cDNA (or portion thereof) is cloned into the vector in the antisense orientation. The antisense expression vector can be in the form of, for example, a recombinant plasmid, phagemid or attenuated virus. The antisense expression vector is introduced into cells using a standard transfection technique, as described above for recombinant expression vectors.

In another embodiment, an antisense nucleic acid for use as an inhibitory agent is a ribozyme. Another type of inhibitory agent that can be used to inhibit the expression and/or activity of human MEKK2 in a cell is an intracellular antibody specific for the human MEKK2 protein. The use of intracellular antibodies to inhibit protein function in a cell is known in the art. To inhibit protein activity using an intracellular antibody, a recombinant expression vector is prepared which encodes the antibody chains in a form such that, upon introduction of the vector into a cell, the antibody chains are expressed as a functional antibody in an intracellular compartment of the cell. For inhibition of human MEKK2 activity according to the inhibitory methods of the invention, an intracellular antibody that specifically binds the human MEKK2 protein is expressed in the cytoplasm of the cell. To inhibit human MEKK2 activity in a cell, the expression vector encoding the anti-human MEKK2 intracellular antibody is introduced into the cell by standard transfection methods, as discussed hereinbefore.

Yet another form of an inhibitory agent of the invention is an inhibitory form of human MEKK2, also referred to herein as a dominant negative inhibitor. The MEKK2 proteins are known to modulate the activity of MEKK2 target molecules, particularly by modulating the phosphorylation state of the MEKK2 target molecule. One means to inhibit the activity of molecule that has an enzymatic activity is through the use of a dominant negative inhibitor that has the ability to interact with the target molecule but that lacks enzymatic activity. By interacting with the target molecule, such dominant negative inhibitors can inhibit the activation of the target molecule. This process may occur naturally as a means to regulate enzymatic activity of a cellular signal transduction molecule.

Accordingly, an inhibitory agent of the invention can be a form of a human MEKK2 protein that has the ability to interact with other proteins but that lacks enzymatic activity. This dominant negative form of a human MEKK2 protein may be, for example, a mutated form of human MEKK2 in which a kinase domain consensus sequence has been altered. Such dominant negative human MEKK2 proteins can be expressed in cells using a recombinant expression vector encoding the human MEKK2 protein, which is introduced into the cell by standard transfection methods. The mutated DNA is inserted into a recombinant expression vector, which is then introduced into a cell to allow for expression of the mutated human MEKK2, lacking enzymatic activity.

Another means to inhibit the activity of the MEKK2 proteins of the invention is via RNA interference (RNAi) (see e.g., Elbashir et al. (2001) Nature 411:494-498 and Elbashir et al. (2001) Genes Development 15:188-200). RNAi is the process of sequence-specific, post-transcriptional gene silencing, initiated by double-stranded RNA (dsRNA) that is homologous in sequence to the silenced gene (i.e., is homologous in sequence to the MEKK2 set forth as SEQ ID NO: 1). siRNA-mediated silencing is thought to occur post-transcriptionally and/or transcriptionally. For example, siRNA duplexes may mediate post-transcriptional gene silencing by reconstitution of siRNA-protein complexes (siRNPs), which guide mRNA recognition and targeted cleavage.

Accordingly, another form of an inhibitory agent of the invention is a small interfering RNA (siRNA) directed against MEKK2. Exemplary siRNAs are 21-nt siRNA duplexes having a sequence homologous or identical to the MEKK2 sequence set forth as SEQ ID NO:1, and having a symmetric 2-nt 3′ overhang. Preferred siRNAs have a sequence homologous or identical to MEKK2 coding sequence (SEQ ID NO:3). The 2-nucleotide 3′ overhang is preferably composed of (2′-deoxy) thymidine because it reduces costs of RNA synthesis and may enhance nuclease resistance of siRNAs in the cell culture medium and within transfected cells. Substitution of uridine by thymidine in the 3′ overhang is also well tolerated in mammalian cells, and the sequence of the overhang appears not to contribute to target recognition.

Other inhibitory agents that can be used to inhibit the activity of a human MEKK2 protein are chemical compounds that directly inhibit human MEKK2 activity or inhibit the interaction between human MEKK2 and target molecules. Such compounds can be identified using screening assays that select for such compounds, as described in detail above.

B. Stimulatory Agents

According to a modulatory method of the invention, human MEKK2 activity is stimulated in a cell by contacting the cell with a stimulatory agent. Examples of such stimulatory agents include active human MEKK2 protein and nucleic acid molecules encoding human MEKK2 that are introduced into the cell to increase human MEKK2 activity in the cell. A preferred stimulatory agent is a nucleic acid molecule encoding a human MEKK2 protein, wherein the nucleic acid molecule is introduced into the cell in a form suitable for expression of the active human MEKK2 protein in the cell. To express a human MEKK2 protein in a cell, typically a human MEKK2-encoding DNA is first introduced into a recombinant expression vector using standard molecular biology techniques, as described herein. A human MEKK2-encoding DNA can be obtained, for example, by amplification using the polymerase chain reaction (PCR), using primers based on the human MEKK2 nucleotide sequence. Following isolation or amplification of human MEKK2-encoding DNA, the DNA fragment is introduced into an expression vector and transfected into target cells by standard methods, as described herein.

Other stimulatory agents that can be used to stimulate the activity of a human MEKK2 protein are chemical compounds that stimulate human MEKK2 activity in cells, such as compounds that directly stimulate human MEKK2 protein and compounds that promote the interaction between human MEKK2 and target molecules. Such compounds can be identified using screening assays that select for such compounds, as described in detail above.

The modulatory methods of the invention can be performed in vitro (e.g, by culturing the cell with the agent or by introducing the agent into cells in culture) or, alternatively, in vivo (e.g., by administering the agent to a subject or by introducing the agent into cells of a subject, such as by gene therapy). For practicing the modulatory method in vitro, cells can be obtained from a subject by standard methods and incubated (i.e., cultured) in vitro with a modulatory agent of the invention to modulate human MEKK2 activity in the cells. If desired, cells treated in vitro with a modulatory agent of the invention can be re-administered to the subject. Preferred cells for in vitro treatment are immune cells. Particularly preferred cells for in vitro treatment include, but are not limited to, basophils, eosinophils, and the like.

For administration to a subject, it may be preferable to first remove residual agents in the culture from the cells before administering them to the subject. For practicing the modulatory method in vivo in a subject, the modulatory agent can be administered to the subject such that human MEKK2 activity in cells of the subject is modulated. The term “subject” is intended to include living organisms in which a MEKK2-dependent cellular response can be elicited. Preferred subjects are mammals. Examples of subjects include humans, monkeys, dogs, cats, mice, rats, cows, horses, goats and sheep. A preferred subject is a human subject. A particularly preferred human subject is humans having an immune disorder, for example, a human subject having asthma.

For stimulatory or inhibitory agents that comprise nucleic acids (including recombinant expression vectors encoding human MEKK2 protein, antisense RNA, intracellular antibodies or dominant negative inhibitors), the agents can be introduced into cells of the subject using methods known in the art for introducing nucleic acid (e.g., DNA) into cells in vivo. Examples of such methods encompass both non-viral and viral methods, including: direct injection, administration via cationic lipids, administration via receptor-mediated DNA uptake, retroviral administration, adenoviral administration and adeno-associated viral administration.

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection or reverse transcriptase-polymerase chain reaction (RT-PCR). The gene product can be detected by an appropriate assay, for example by immunological detection of a produced protein, such as with a specific antibody, or by a functional assay to detect a functional activity of the gene product.

In a preferred embodiment, a retroviral expression vector encoding human MEKK2 is used to express human MEKK2 protein in cells in vivo, to thereby stimulate MEKK2 protein activity in vivo. Such retroviral vectors can be prepared according to standard methods known in the art (discussed further above).

A modulatory agent, such as a chemical compound, can be administered to a subject as a pharmaceutical composition. Such compositions typically comprise the modulatory agent and a pharmaceutically acceptable carrier. As used herein the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

This invention is further illustrated by the following example, which should not be construed as limiting. The contents of all references, patents and published patent applications cited throughout this application are hereby incorporated by reference. Additionally, all nucleotide and amino acid sequences deposited in public databases referred to herein are also hereby incorporated by reference.

### EXEMPLIFICATION

### Example 1

### Isolation and Cloning of Human MEKK2 Nucleic Acid

Total RNA from human acute T-cell leukemia cells (Jurkat E6-1; ATCC TIB-152) was extracted using TriZol™ Reagent and further purified by RNA purification minicolumn (Qiagen). Message RNA (mRNA) was reverse transcribed following a modified version of a standard reverse transcriptase reaction using MMLV/RNAse H-Reverse Transcriptase (PowerScript™; Clontech) and oligo(dT)₁₂₋₁₈. The resulting cDNA collection served as a template for the amplification of 1.2 kB 5′ fragment and 1.0 kB 3′fragment using high-fidelity, proofreading thermostable DNA polymerase (Pfu Turbo™; Stratagene), standard PCR conditions and the following sets of primers were used.

| | |
|---|---|
| 5′ fragment; | |
| 5′ Forward | |
| ACGGCCATAAAGAATGCTGATT, | (SEQ ID NO:13) |
| 5′ Reverse | |
| TGCCCAATCTCCAGTTGGTC; | (SEQ ID NO:14) |
| 3′ fragment; | |
| 3′ Forward | |
| AGAAGGGGAAGTGACATAGACAAT | (SEQ ID NO:15) |
| 3′ Reverse | |
| TAACAATTTAACCAAGAATCAAGA. | (SEQ ID NO:16) |

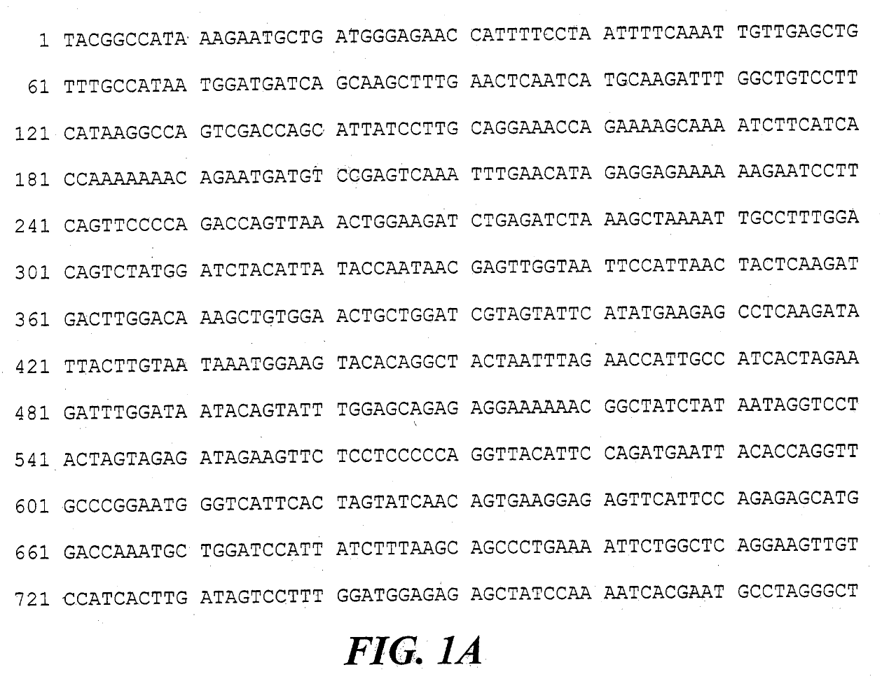

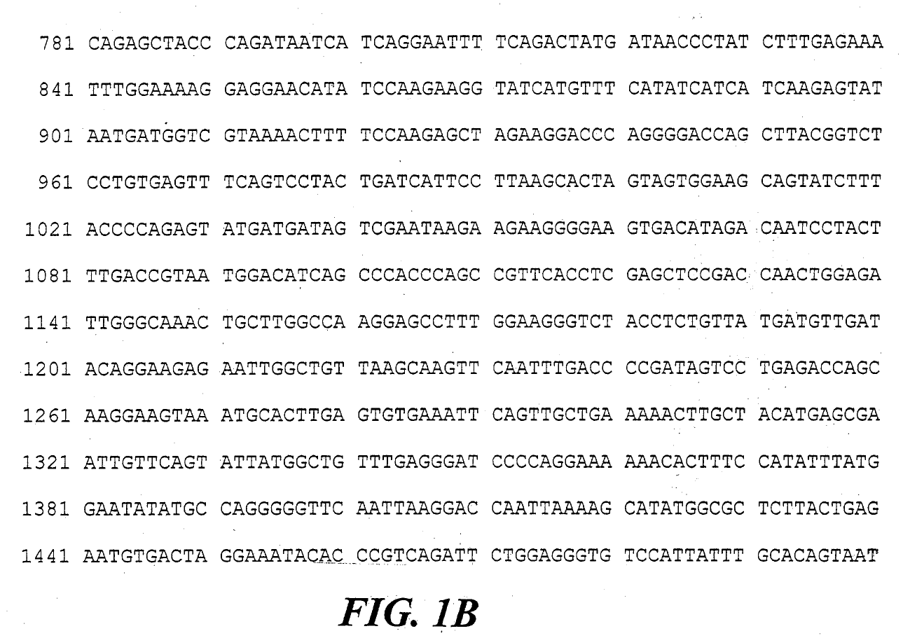

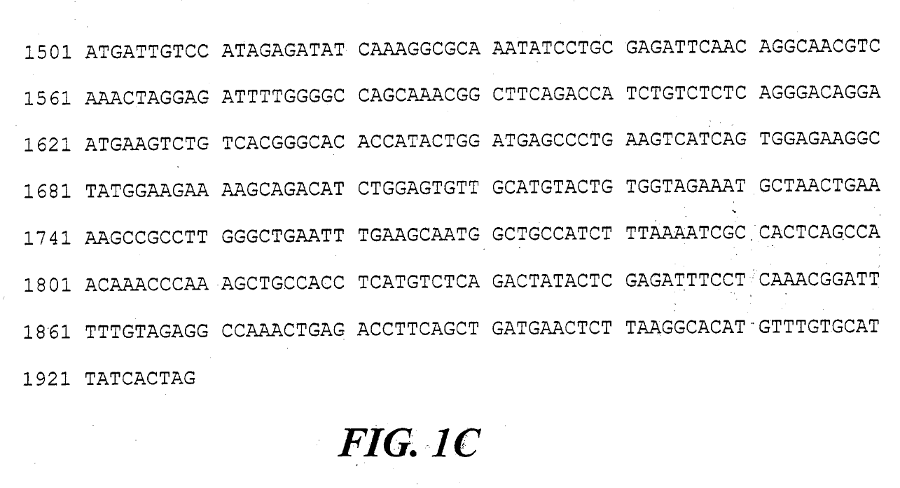

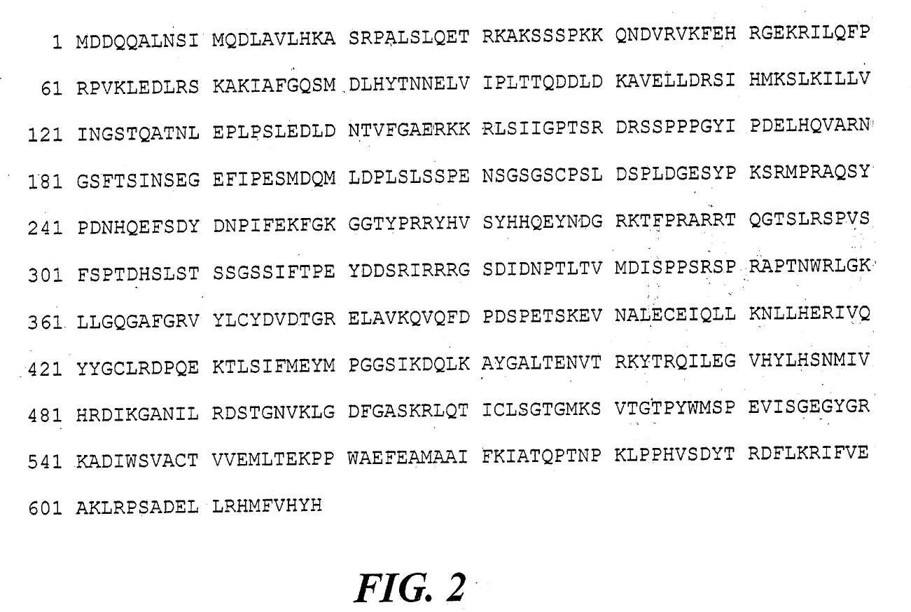

The two amplicons of expected size were purified by PCR column purification. The MEKK2 partial products were re-amplified using Pfu Turbo™ and original MEKK2 5′Forward and MEKK2 3′Reverse primers (described above) to splice the intermediate products together via a PCR-based primer extension strategy which used the overlapping homologous sequence as the initiating extension primers. A 1.9 kb product was cloned into pTA-NT vector™ (Invitrogen) for sequencing verification. Sequence information was obtained using standard fluorescent-based dye terminator technology using an ABI 377 sequencer. Sequence discrepancies were resolved by comparison of sequence information from complementary strands, as well as comparison of automated base-call to the original electropherogram. Sequence of various read lengths from the plus and minus strands were then assembled in contiguous fashion using overlap reads as guides, based on sequence alignment and analysis in Martinez/Needleman-Wunsch alignment method available in DNAStar™ DNA analysis software. The result was a single sequence for the entire 1.9 kB product. The MEKK2 nucleotide sequence is depicted in FIGS. 1A-C and set forth as SEQ ID NO:1. The coding region is from residues 70-1870. The 5′ untranslated region is from residues 1-69 of SEQ ID NO:1. Coding region only is set forth as SEQ ID NO:3. The MEKK2 amino acid sequence is depicted in FIG. 2 and set forth as SEQ ID NO:2.

The following sequence primers were used to verify the authenticity of the MEKK2 clones from Jurkats, spleen and thymus:

| | |
|---|---|
| AAACAAGTTCAATTTGAC | (SEQ ID NO:17) |
| ATCGGGGTCAAATTGAAC | (SEQ ID NO:18) |
| ACCAATAACGCGTTGGTA | (SEQ ID NO:19) |
| TCACAGGAGACGTAAGCT | (SEQ ID NO:20) |
| AGCCAAATCTTGCATGATT | (SEQ ID NO:21) |
| ACTATACTCGAGATTTCCT | (SEQ ID NO:22) |
| ATGGATGATCAGCAAGCT | (SEQ ID NO:23) |
| ATTATACCAATAACGAGT | (SEQ ID NO:24) |
| TCTATAATAGGTCCTACT | (SEQ ID NO:25) |
| CAAAATCACGAATGCCTA | (SEQ ID NO:26) |
| ACAATCCTACTTTGAACC | (SEQ ID NO:27) |
| ACTTTCCATATTTATGGA | (SEQ ID NO:28) |
| TGGCAGCTTTGGGTTTGT | (SEQ ID NO:29) |
| TCTGACGGGTGTATTTC | (SEQ ID NO:30) |
| ACTCACAGGAGACCGTAA | (SEQ ID NO:31) |
| ATGGCAATGGTTCTAAAT | (SEQ ID NO:32) |

### Example 2

### Verification of Unique Form of MEKK2 from Spleen and Thymus

Based on the protocol outlined above, a 1.2 kB fragment and a 1.0 kB fragments were amplified from total RNA from both non-disease human thymus and non-disease human spleen (Ambion). Amplicons from each source were cloned into a sequencing vector, pBLUNT™ (Invitrogen). Sequencing results confirmed that human MEKK2 overlapping fragments from thymus and spleen was found to be identical to human MEKK2 representative clones isolated from Jurkat cells.

### Example 3

### Sequence Comparison to Published Forms of MEKK2

The MEKK2 DNA sequence described in Example 2 was searched against DNA sequences appearing in the Genbank™, CGAP™, and other public DNA sequence databases using the Basic Local Alignment Search Tool, BLASTN™, available from the NCBI public website. The MEKK2 amino acid sequence was likewise searched against protein sequences appearing in the public databases using the Basic Local Alignment Search Tool, BLASTP™, available from the NCBI public website.

Comparisons of selected MEKK2 sequences found in the public databases were performed using Martinez/Needleman-Wunsch alignment for DNA, and Lipman-Pearson method for putative protein alignments, available in DNAStar™ Software Package. Additional protein comparisons were performed using the LALIGN alignment algorithm (see e.g., Huang and Miller (1991) Adv. Appl. Math. 12:337-357) using a PAM120 weight residue table, gap penalties −2/−12. The LAIGN algorithm is freely available at the EMBnet website maintained by the Swiss Institute of Bioinformatics. Multiple sequence alignments were performed using the ClustalW alignment algorithm. The ClustalW algorithm is freely available at the GenomeNet website maintained by the Bioinformatics Center of the Institute for Chemical Research, Kyoto University

FIGS. 3A-C depicts an alignment of the MEKK2 protein described in Example 2 with four previously-identified MEKK2 isoforms. The alignment was generated using the ClustalW alignment algorithm. Shading indicates identical amino acid residues and bold indicates homologous amino acid residues. The kinase domain is indicated by arrows. Box I indicates the “protein kinase ATP-binding region signature”. The ATP-binding lysine is indicated by an asterisk. Box II indicates the “serine/threonine protein kinase active-site signature”. The active site aspartate residue is indicated by an asterisk. The isoforms are as follows. MEKK2.1a is an isoform described by Su and Karin (direct submission) on GenBank having Accession No. AAD28547 (SEQ ID NO:5). The corresponding nucleotide sequence has accession No. AF111105 (SEQ ID NO:4). MEKK2.1b is an isoform described by Su et al. (2001) J. Biol. Chem. 276:14784-14790 (SEQ ID NO:6). This isoform is depicted in FIG. 1A of the reference and the legend states that the isoform is the same as that deposited in GenBank under Accession No. AF111105. Notably, there are discrepancies between the Su and Karin GenBank record and the sequence published in J. Biol. Chem. MEKK2.2 is an isoform described in U.S. Pat. No. 6,312,934 by Johnson (amino acid sequence set forth as SEQ ID NO:7, amino acid sequence set forth as SEQ ID NO:8). MEKK2.3 is an isoform described by Wang and Lo (Viogenics, Sunnyvale, Calif.) (direct submission) having GenBank Accession No. AAF63496 (SEQ ID NO: 10). The corresponding nucleotide sequence has accession No. AF239798 (SEQ ID NO:9). MEKK2.4 is the MEKK2 amino acid sequence described in Example 2 (SEQ ID NO:2).

Theoretical pI calculations were made in ExPASy Server based on denaturing conditions described by Bjellqvist et al. (1993) Electrophoresis 14:1023-31.

### Example 4

### Preparation of Recombinant Human MEKK2 Proteins

Different MEKK2-fusion proteins have been cloned for expression and detection of the protein in bacteria, baculovirus-infected SF-9 cells or mammalian cells. A polyhistidine-hMEKK2 fusion was created as follows: hMEKK2 was amplified by PCR with the following primers, 5′His-Forward and 3′-Reverse. The resulting amplicon encoding for a 6×HIS tag at the N-terminus, followed by the methionine and complete sequence of hMEKK2 was purified and ligated into pQE-TriSystem™ (Qiagen) vector (which contains all the appropriate signals for efficient expression of the fusion protein in bacteria, baculovirus-infected Sf9 or Sf21 cells, and diverse mammalian systems). Clones were selected from transformed E. coli TOP10F′ competent cells and sequence-verified as outlined above.

For production of a Glutathione-S-transferase (GST)-fusion, hMEKK2 was ligated in frame to the multiple cloning region of baculovirus vector pAcG2T, downstream from the GST coding region (BD Bioscience) and transformed into E. coli strain TOP10F′ competent cells. Positive clones for GST-hMEKK2 fusion were identified using standard molecular biology techniques, and then sequence-verified.

pQE-His-tagged-hMEKK2 was transfected into Sf9 insect cells and baculoviral stocks were produced. The baculovirus was further purified and characterized by plaque purification. Plaque-purified clonal baculovirus isolates were tested for infectivity and expression of His-tagged hMEKK2. A Western blot of infected Sf9 cells extracts revealed that over half of the clonal virus plaques examined expressed high levels of a His-tag mAb antibody-reactive protein band at the expected size for the His-hMEKK2. Kinase reactions were performed on each extract to determine the extent of enzymatic activity of the hMEKK2 produced in each extract. In these assays, 1 λ of Sf9 crude extract expressing either His-tagged-hMEKK2 or control Sf9 extract was mixed with kinase buffer (20 mM HEPES, pH 7.5, 5 mM MgCl2, 1 mM DTT) containing 10 μCi [γ-³²P]-ATP (6 Ci/μmol; New England Nuclear) and 250 ng of GST-MKK4 fusion protein (available from Upstate Biotechnologies, Inc) in a final volume of 20 λ. The reactions were incubated at 30° C. for 30 mins and were terminated by addition of 20 λ SDS-loading buffer. Reaction components were separated on SDS-12% polyacrylamide gel under denaturing conditions and exposed using BioRad Molecular Imager FX. The ability of extracts containing his-tagged hMEKK2 to phosphorylate GST-MKK4 was comparable to the activity of purified mouse MEKK2, and significantly above the background levels seen with control extracts. In addition, ³²P incorporated was observed for the His-hMEKK2 protein itself, indicative of its ability Lo autophosporylate and generate unique phosphorylated forms of hMEKK2.

Equivalents

Those skilled in the art will recognize, or be able to ascertain using no more than routine experimentation, many equivalents to the specific embodiments of the invention described herein. Such equivalents are intended to be encompassed by the following claims.

We claim:

1. An isolated nucleic acid molecule comprising a nucleotide sequence encoding a MEKK2 protein, said MEKK2 protein having an amino acid sequence at least 99.1% identical to the amino acid sequence of SEQ ID NO:2, wherein % identity is determined according to the LALIGN algorithm using a PAM120 weight residue table, a gap length penalty of 12 and a gap penalty of 4.

2. The nucleic acid molecule of claim 1, which encodes a MEKK2 protein having an amino acid sequence at least 99.5% identical to the amino acid sequence of SEQ ID NO:2.

3. The nucleic acid molecule of claim 1, which encodes a MEKK2 protein having an amino acid sequence at least 99.9% identical to the amino acid sequence of SEQ ID NO:2.

4. An isolated nucleic acid molecule which encodes a MEKK2 protein comprising the amino acid sequence of SEQ ID NO:2.

5. An isolated nucleic acid molecule which comprises the nucleotide sequence of SEQ ID NO:3.

6. An isolated nucleic acid molecule encoding a MEKK2 protein comprising amino acids 356-616 of SEQ ID NO:2.

7. An isolated nucleic acid molecule encoding a MEKK2 fragment consisting essentially of amino acids 356-616 of SEQ ID NO:2.

8. An isolated nucleic acid molecule encoding a fusion protein comprising a MEKK2 fragment consisting essentially of amino acids 356-616 of SEQ ID NO:2 and at least one non-MEKK2 polypeptide.

9. A vector comprising the nucleic acid molecule of any one of claims 1-2 and 4-8.

10. The vector of claim 9, which is an expression vector.

11. A host cell containing the vector of claim 10.

12. A method for producing a MEKK2 protein comprising culturing the host cell of claim 11 in a suitable medium until a MEKK2 protein is produced.

13. The method of claim 12, further comprising isolating the MEKK2 protein from the medium or the host cell.

14. An isolated MEKK2 protein comprising an amino acid sequence at least 99.1% identical to the amino acid sequence of SEQ ID NO:2, wherein % identity is determined according to the LALIGN algorithm using a PAM120 weight residue table, a gap length penalty of 12 and a gap penalty of 4.

15. An isolated protein comprising the amino acid sequence of SEQ ID NO:2.

16. The protein of claim 14, which comprises an amino acid sequence at least 99.5% identical to the amino acid sequence of SEQ ID NO:2.

17. The protein of claim 14, which comprises an amino acid sequence at least 99.9% identical to the amino acid sequence of SEQ ID NO:2.

18. An isolated protein encoded by a nucleic acid molecule comprising the nucleotide sequence of SEQ ID NO3.

19. An isolated protein comprising amino acids 356-616 of SEQ ID NO:2.

20. An isolated MEKK2 fragment consisting essentially of amino acids 356-616 of SEQ ID NO:2.

21. A fusion protein comprising a MEKK2 fragment consisting essentially of amino acids 356-616 of SEQ ID NO:2 and at least one non-MEKK2 polypeptide.

22. A fusion protein comprising the MEKK2 protein of any one of claims 14-15 and 17-18, operatively linked to a polypeptide other than MEKK2.

23. The MEKK2 protein of any one of claims 14-19, wherein said protein is phosphorylated.

24. The MEKK2 protein of claim 23, wherein said protein includes at least 1 mole of phosphate per mole of protein.

25. The MEKK2 protein of claim 23, wherein said protein includes at least 2 moles of phosphate per mole of protein.

26. An isolated antibody that specifically binds to the MEKK2 protein of claim 14.

27. An isolated antibody that specifically binds to the MEKK2 protein of claim 23.

28. The antibody of claim 26, which is a polyclonal antibody.

29. The antibodies of claim 26, which is a monoclonal antibody.

30. The antibody of claim 26, which is coupled to a detectable substance.

31. The antibody of claim 27, which is a polyclonal antibody.

32. The antibodies of claim 27, which is a monoclonal antibody.

33. The antibody of claim 27, which is coupled to a detectable substance.

34. A nonhuman transgenic animal that contains cells carrying a transgene that encodes the MEKK2 protein of claim 14.

35. A nonhuman transgenic animal that having the gene encoding the MEKK2 protein set forth as SEQ ID NO:2 mutated or deleted such that the animal fails to express the MEKK2 protein.

36. A method for specifically detecting the presence the MEKK2 protein of claim 11 in a biological sample comprising contacting the biological sample with an agent capable of specifically detecting the MEKK2 protein such that the presence of the MEKK2 protein is detected in the biological sample.

37. A method for specifically detecting the presence the nucleic acid molecule of claim 1 in a biological sample comprising contacting the biological sample with an agent capable of specifically detecting the nucleic acid molecule such that the presence of the nucleic acid molecule is detected in the biological sample.

38. A method for identifying a MEKK2 modulator comprising contacting the MEKK2 protein of claim 14 with a test compound; determining whether the compound modulates an activity of the MEKK2 protein; and identifying the compound as a MEKK2 modulator where the compound modulates the activity of the MEKK2 protein.

39. A method for identifying a MEKK2 modulator comprising contacting the MEKK2 protein of claim 14 with a test compound and determining the effect of the test compound on an activity of the MEKK2 protein to thereby identify the compound as a MEKK2 modulator.

40. A method for identifying a MEKK2 modulator comprising contacting a cell which expresses the MEKK2 protein of claim 14 with a test compound and determining the effect of the test compound on an activity of the MEKK2 protein to thereby identify the compound as a MEKK2 modulator.

41. The method of claim 39 or 40, wherein determining the effect of the test compound on an activity of the MEKK2 protein comprises determining a stimulatory effect.

42. The method of claim 39 or 40, wherein determining the effect of the test compound on an activity of the MEKK2 protein comprises determining an inhibitory effect.

43. The method of claim 40, wherein the cell further comprises a reporter gene responsive to the MEKK2 protein and determining whether the compound modulates an activity of the MEKK2 protein comprises determining the level of expression of the reporter gene in the presence of the test compound to thereby identify the MEKK2 modulator.

44. A method for identifying a MEKK2 modulator comprising contacting a cell which expresses the MEKK2 nucleic acid molecule of claim 1 with a test compound and determining the effect of the test compound on expression of the MEKK2 nucleic acid molecule to thereby identify the compound as a MEKK2 modulator.

45. The method of claim 44, wherein determining the effect of the test compound on expression of the MEKK2 nucleic acid molecule comprises determining a stimulatory effect.

46. The method of claim 44, wherein determining the effect of the test compound on expression of the MEKK2 nucleic acid molecule comprises determining an inhibitory effect.

47. A method for identifying a MEKK2-specific modulator comprising:
(a) contacting a MEKK2 protein or cell expressing said MEKK2 protein with a test compound;
(b) determining the effect of the test compound on the expression or activity of the MEKK2;
(c) contacting a MEKK1, MEKK3 or MEKK4 protein or cell expressing said MEKK1, MEKK3 or MEKK4 protein with said test compound;
(d) determining the effect of the test compound on the expression or activity of the MEKK1, MEKK3 or MEKK4 protein;
(e) comparing the effect as determined in subpart (b) with the effect as determined in subpart (d); and
(f) identifying the compound as a MEKK2-specific modulator where said compound exhibits an effect as determined in subpart (b) and exhibits no effect as determined in subpart (d).

48. The method of any one of claims 38-40 and 44, wherein the test compound is selected from the group consisting of an antisense nucleic acid molecule, a peptide, a peptidomimetic or a small molecule.

49. The method of claim 39 or 40, wherein the activity is selected from the group consisting of (i) interaction of a MEKK2 protein with a MEKK2 binding partner, wherein the binding partner effects the activity of the MEKK2 molecule; (ii) interaction of a MEKK2 protein with a MEKK2 target molecule, wherein the MEKK2 protein effects the activity of the target molecule; (iii) phosphorylation of a MEKK2 target molecule selected from the group consisting of MKK1, MKK2, MKK3, MKK4, MKK5, MKK6, and MKK7; (iv) phosphorylation of a non-target protein; (v) mediation of activation of MAPK signal transduction molecules; (vi) modulation of the activity of a nuclear transcription factor; and (vii) modulation of ERK-, JNK- or p38-dependent gene transcription.

50. The method of claim 39 or 40, wherein the activity is selected from the group consisting of (i) modulation of cellular signal transduction, either in vitro or in vivo; (ii) regulation of gene transcription in a cell expressing a MEKK2 protein; (iii) regulation of gene transcription in a cell expressing a MEKK2 protein, wherein said cell is involved inflammation; (iv) regulation of cellular proliferation; (v) regulation of cellular differentiation; (vi) regulation of development; (vii) regulation of cell death; and (viii) regulation of inflammation.

51. Use of the MEKK2 protein set forth as SEQ ID NO:2 in a method for identifying a compound which specifically modulates the expression or activity of a non-MEKK2 protein, wherein a lack of expression or activity of said MEKK2 protein as compared to the expression or activity of the non-MEKK2 protein indicates that the compound specifically modulates the expression or activity of the non-MEKK2 protein.

52. A method for modulating a MEKK2 activity in a cell comprising contacting the cell with the MEKK2 modulator identified by the method of any one of claims 38-40 and 44, such that the MEKK2 activity in the cell is modulated.
